SEQUENCE LISTING (53 sequences)

SEQ ID NO: 1 - DNA, 32 bp, Artificial sequence
  DNAzyme sequence
gcctgttgggctagctacaacgacagatattt

SEQ ID NO: 2 - DNA, 33 bp, Artificial sequence
  DNAzyme sequence
gttgtcagaggctagctacaacgaatttttccg

SEQ ID NO: 3 - DNA, 33 bp, Artificial sequence
  DNAzyme sequence
ccagatccaggctagctacaacgattctatcat

SEQ ID NO: 4 - DNA, 32 bp, Artificial sequence
  DNAzyme sequence
tagagtaagggctagctacaacgaagttcgcc

SEQ ID NO: 5 - DNA, 33 bp, Artificial sequence
  DNAzyme sequence
ttttagttgggctagctacaacgagttagtact

SEQ ID NO: 6 - DNA, 35 bp, Artificial sequence
  DNAzyme sequence
tattttagcaggctagctacaacgaagtcatttaa

SEQ ID NO: 7 - DNA, 35 bp, Artificial sequence
  DNAzyme sequence
tgactcataaggctagctacaacgatattttagca

SEQ ID NO: 8 - DNA, 37 bp, Artificial sequence
  DNAzyme sequence
tgactcataatggctagctacaacgatttagcatagt

SEQ ID NO: 9 - DNA, 33 bp, Artificial sequence
  DNAzyme sequence
gcttttttaggctagctacaacgatgactaatg

SEQ ID NO: 10 - DNA, 35 bp, Artificial sequence
  DNAzyme sequence
agcttttttaggctagctacaacgatgactaatgg

SEQ ID NO: 11 - DNA, 33 bp, Artificial sequence
  DNAzyme sequence
ttagcatagggctagctacaacgacatttaaat

SEQ ID NO: 12 - DNA, 37 bp, Artificial sequence
  DNAzyme sequence
ttttagcatagggctagctacaacgacatttaaataa

SEQ ID NO: 13 - DNA, 33 bp, Artificial sequence
  DNAzyme sequence
attttagcaggctagctacaacgaagtcattta

SEQ ID NO: 14 - DNA, 37 bp, Artificial sequence
  DNAzyme sequence
ttattttagcaggctagctacaacgaagtcatttaaa

SEQ ID NO: 15 - DNA, 33 bp, Artificial sequence
  DNAzyme sequence
gactcataaggctagctacaacgatattttagc

SEQ ID NO: 16 - DNA, 33 bp, Artificial sequence
  DNAzyme sequence
tttagtaagggctagctacaacgatctttatgg

SEQ ID NO: 17 - DNA, 35 bp, Artificial sequence
  DNAzyme sequence
atttagtaagggctagctacaacgattatgggtac

SEQ ID NO: 18 - DNA, 33 bp, Artificial sequence; identical to SEQ ID NO: 5
  DNAzyme sequence

SEQ ID NO: 19 - DNA, 35 bp, Artificial sequence
  DNAzyme sequence
tttttagttgggctagctacaacgagttagtactc

SEQ ID NO: 20 - DNA, 33 bp, Artificial sequence
  DNAzyme sequence
tcataattaggctagctacaacgatttagcata

SEQ ID NO: 21 - DNA, 33 bp, Artificial sequence
  DNAzyme sequence
gaagtcgtgggctagctacaacgacagatacat

SEQ ID NO: 22 - DNA, 31 bp, Artificial sequence
  DNAzyme sequence
gttcgttgtccgagccggacgacgaataatt

SEQ ID NO: 23 - DNA, 33 bp, Artificial sequence; identical to SEQ ID NO: 9
  DNAzyme sequence

SEQ ID NO: 24 - DNA, 33 bp, Artificial sequence
  DNAzyme sequence
atttatttaggctagctacaacgaaaaatttac

SEQ ID NO: 25 - DNA, 33 bp, Artificial sequence
  DNAzyme sequence
tttttcgtgggctagctacaacgattctttttc

SEQ ID NO: 26 - DNA, 33 bp, Artificial sequence; identical to SEQ ID NO: 20
  DNAzyme sequence

SEQ ID NO: 27 - DNA, 18 bp, Artificial sequence
  target region for Dnzyme KPC_337
aaatatctgacaacaggc

SEQ ID NO: 28 - DNA, 15 bp, Artificial sequence
  catalytic domain of 10-23 DNAzymes
ggctagctacaacga

SEQ ID NO: 29 - DNA, 14 bp, Artificial sequence
  catalytic domain of 8-17 DNAzymes
tccgagccggacga

SEQ ID NO: 30 - DNA, 11 bp, Artificial sequence
  16.2-11 catalytic core
gtgaccccuug

SEQ ID NO: 31 - DNA, 18 bp, Artificial sequence
  9-86 catalytic core
ucaugcagcgcguagugu

SEQ ID NO: 32 - DNA, 18 bp, Artificial sequence
  the 12-91 catalytic core
ugaugcagcgcauguguc

SEQ ID NO: 33 - DNA, 13 bp, Artificial sequence
  FR17_6 catalytic core
aagcaguuaagac

SEQ ID NO: 34 - DNA, 22 bp, Artificial sequence
  Bipartite I or Bipartite II catalytic core
aaggaggtaggggttccgctcc

SEQ ID NO: 35 - DNA, 33 bp, Artificial sequence
  DNAzyme sequence
cagtttttgggctagctacaacgaaagctttcc

SEQ ID NO: 36 - DNA, 33 bp, Artificial sequence
  DNAzyme sequence
catgagagaggctagctacaacgaaagacagca

SEQ ID NO: 37 - DNA, 31 bp, Artificial sequence
  DNAzyme sequence
gaacgtggggctagctacaacgaatcgccga

SEQ ID NO: 38 - DNA, 32 bp, Artificial sequence
  DNAzyme sequence
cggcgttaggctagctacaacgacactgtatt

SEQ ID NO: 39 - DNA, 33 bp, Artificial sequence
  DNAzyme sequence
ttttgtaagggctagctacaacgatttccgtca

SEQ ID NO: 40 - DNA, 33 bp, Artificial sequence; identical to SEQ ID NO: 2
  DNAzyme sequence

SEQ ID NO: 41 - DNA, 33 bp, Artificial sequence
  DNAzyme sequence
cagtgtcagggctagctacaacgattttgtaag

SEQ ID NO: 42 - DNA, 31 bp, Artificial sequence
  DNAzyme sequence
gtgtttcctccgagccggacgattagccaat

SEQ ID NO: 43 - DNA, 32 bp, Artificial sequence
  DNAzyme sequence
ccgtcatgggctagctacaacgactgttgtca

SEQ ID NO: 44 - DNA, 33 bp, Artificial sequence
  DNAzyme sequence
atagacttgggctagctacaacgattgtatgtg

SEQ ID NO: 45 - DNA, 32 bp, Artificial sequence
  DNAzyme sequence
ccacggaaggctagctacaacgatgattggga

SEQ ID NO: 46 - DNA, 33 bp, Artificial sequence
  DNAzyme sequence
tataaggtaggctagctacaacgattccggtaa

SEQ ID NO: 47 - DNA, 32 bp, Artificial sequence
  DNAzyme sequence
tttggcgaggctagctacaacgatgcaagatt

SEQ ID NO: 48 - DNA, 33 bp, Artificial sequence
  DNAzyme sequence
ccatttctaggctagctacaacgacatgcctac

SEQ ID NO: 49 - DNA, 33 bp, Artificial sequence
  DNAzyme sequence
ggtggctaaggctagctacaacgaagggagata

SEQ ID NO: 50 - DNA, 33 bp, Artificial sequence
  DNAzyme sequence
ttaaaggtgggctagctacaacgatcatcatgg

SEQ ID NO: 51 - DNA, 32 bp, Artificial sequence
  DNAzyme sequence
gctcgtcgggctagctacaacgattggtatgg

SEQ ID NO: 52 - DNA, 33 bp, Artificial sequence
  DNAzyme sequence
tctatttcgggctagctacaacgatcatccata

SEQ ID NO: 53 - DNA, 33 bp, Artificial sequence
  DNAzyme sequence
acacggaaaggctagctacaacgagttgaatac

(19) United States
(12) Patent Application Publication
(10) Pub. No.: US 2023/0416750 A1
(43) Pub. Date: Dec. 28, 2023

(54) CATALYTIC SEQUENCE BASED METHODS OF TREATING OR PREVENTING BACTERIAL INFECTIONS

(71) Applicant: 1E Therapeutics Ltd., Rehovot (IL)

(72) Inventors: Ido Bachelet, Rehovot (IL); Almogit Horowitz, Rehovot (IL); Alexander Rosenberg, Rehovot (IL); Anastasia Shapiro, Rehovot (IL); Ron Oshri, Rehovot (IL); Ilana Kolodkin-Gal, Rehovot (IL); Adva Levy-Zamir, Rehovot (IL); Gat Krieger, Rehovot (IL); Ella GILLIS, Rehovot (IL); Shmulik ITTAH, Rehovot (IL)

(73) Assignee: 1E Therapeutics Ltd., Rehovot (IL)

(21) Appl. No.: 18/035,871

(22) PCT Filed: Nov. 9, 2021
(86) PCT No.: PCT/IL2021/051331

§ 371 Date: May 8, 2023

Related U.S. Application Data
(60) Provisional application No. 63/111,118, filed on Nov. 9, 2020.

(51) Int. Cl. C12N 15/113; A61K 45/06; A61K 47/54; A61K 31/7088; A61P 31/04

(52) U.S. Cl. CPC C12N 15/1137; A61K 45/06; A61K 47/554; A61K 31/7088; A61P 31/04; C12Y 305/02006

(57) ABSTRACT

An oligonucleotide is provided. The oligonucleotide comprising a nucleic acid sequence of at least one DNAzyme, the DNAzyme being capable of silencing at least one target gene of a bacteria to thereby render the bacteria susceptible to antibiotic treatment.

### RELATED APPLICATION

This application claims the benefit of priority of U.S. Provisional Patent Application No. 63/111,118 filed on Nov. 9, 2020, the contents of which are incorporated herein by reference in their entirety.

SEQUENCE LISTING STATEMENT The ASCII file, entitled 89144 Sequence Listing.txt, created on 9 Nov. 2021, comprising 10,624 bytes, submitted concurrently with the filing of this application is incorporated herein by reference.

### FIELD AND BACKGROUND OF THE INVENTION

The present invention, in some embodiments thereof, relates to DNAzymes targeting bacterial target genes and, more particularly, but not exclusively, to the use of same for treating or preventing bacterial infections.

Antibiotic Resistant Bacteria

The growing numbers of antimicrobial-resistant pathogens, which are increasingly associated with hospital acquired infections, place a significant burden on global healthcare systems. Antibiotic resistant infections are associated with high mortality and morbidity rates, increased treatment costs, diagnostic uncertainties, and failure of conventional medicine/pharmaceuticals. Recent reports using data from hospital-based surveillance studies as well as from the Infectious Diseases Society of America have begun to refer to a group of nosocomial pathogens as “ESKAPE” pathogens, ESKAPE being an acronym for Gram-positive and Gram-negative species, made up of Enterococcus faecium, Staphylococcus aureus, Klebsiella pneumoniae, Acinetobacter baumannii, Pseudomonas aeruginosa and Enterobacter species, which account for most common life-threatening nosocomial infections [Rice, L. B., The Journal of infectious diseases (2008) 197: 1079-1081; and Rice, L. B., Infect. Control Hosp. Epidemiol. (2010) 31 Suppl 1, S7-10]. Among these, Klebsiella pneumoniae infections are a rising category of ESKAPE pathogen infections and are challenging to eradicate due to their increased antibiotic resistance.

β-Lactamases

The mechanism supporting the genotypic, transferrable and heritable antibiotic resistance are frequently attributed to the bacterial β-lactamases. These enzymes hydrolyse the β-lactam ring of β-lactams, the most commonly used class of antibiotics, inactivating the antibiotic before it reaches its target. Carbapenems are a class of highly effective antibiotic agents from the β-lactam family which is usually reserved for known or suspected multidrug-resistant (MDR) bacterial infections, and can be degraded by carbapenemases (i.e. the most versatile family of β-lactamases). One emerging class of carbapenemases are the Klebsiella pneumoniae carbapenemases (KPCs) which were first identified in 1996. Since then, regional outbreaks of KPC-producing K. pneumoniae (KPC-Kp) have spread internationally [Chen, L. et al., Trends Microbiol. (2014) 22, 686-696].

Antibacterial Agents

One progress in antibacterial treatment is the development of antibacterial antisense oligonucleotides. This is generally described as RNA silencing in bacteria using synthetic nucleic acid oligomer mimetics to specifically inhibit essential gene expression and achieve gene-specific antibacterial effects. Usually the antibacterial antisense oligonucleotides are designed to bind the target mRNA to prevent translation or bind DNA to prevent gene transcription (Bai and Luo., Antisense Antibacterials: From Proof-Of-Concept to Therapeutic Perspectives, In tech open book series (2012) chapter 16: 319-344, DOI: 10.5772/33347).

DNAzymes

DNA enzymes (DNAzymes) are synthetic, catalytically-active DNA molecules that are able to specifically cleave target mRNA without requiring the involvement of cellular mechanisms such as the RNA-Induced Silencing Complex (RISC). DNAzymes have not been reported in nature and are typically generated by in-vitro selection. Moreover, DNAzymes are diverse structurally and mechanistically, and exhibit diverse secondary structures, metal ion dependencies, and catalysis kinetics.

DNAzymes typically consist of a catalytic core flanked by two arms that recognize its RNA target through Watson Crick base pairing and cleave RNA in a specific phosphodiester linkage. DNAzymes are a powerful tool for specific gene therapy due to their high specificity and catalytic efficiency, as they are easy to synthesize and modify, and since they are less sensitive to chemical and enzymatic degradation compared to protein and RNA-based reagents [Huo, W. et al., Biophysics Reports (2020) 6: 256-265].

The therapeutic potential of DNAzymes has been demonstrated in diverse settings including in antimicrobial resistant bacterial infections. Hou et al. [Hou et al., Acta Pharmacologica Sinica (2007) 28: 1775-1782] disclose restoration of the β-lactam oxacillin susceptibility of methicillin-resistant Staphylococcus aureus (MRSA) by targeting the signaling pathway of blaR1-blaZ with the DNAzyme PS-DRz602. Furthermore, Bao et al. [Bao et al., Current Genetics (2021) online doi.org/10.1007/s00294-021-01212-0] disclose the use of dnazymes as a diagnostic and therapeutic agent against antimicrobial resistance (AMR). Specifically, Bao discuss direct targeting of bacterial resistant genes, such as beta-lactamases, and indirect targeting of AMR-associated genes, such the cell division gene (ftsZ), by dnazymes.

### ADDITIONAL RELEVANT ART

Chinese Patent Publication no. 107630010 disclosing DNAzymes for inhibiting plasmid mediated quinolone drug resistant gene qnrD in bacteria.

U.S. Patent Application nos. 2006/223774 and 2004/220123 relate to toxic agents, ribozymes, dnazymes and antisense oligonucleotides which are lethal to pathogens and methods for targeting such agents to a pathogen or pathogen infected cells in order to treat and/or eradicate the infection.

### SUMMARY OF THE INVENTION

According to an aspect of some embodiments of the present invention there is provided an oligonucleotide comprising a nucleic acid sequence of at least one DNAzyme, the DNAzyme being capable of silencing at least one target gene of a bacteria selected from the group consisting of:
ESBL: Extended-spectrum beta-lactamase,
Carbapenemase: (5R)-carbapenem-3-carboxylate synthase: EC: 1.14.20.3,
USA300HOU,
mecA: penicillin-binding protein 2A,
mecR1: beta-lactamase-sensing transmembrane signaling protein,
glpT: glycerol-3-phosphate transporter, and
femA: aminoacyltransferase: EC: 2.3.2.17 to thereby render the bacteria susceptible to antibiotic treatment.

According to an aspect of some embodiments of the present invention there is provided a nucleic acid construct encoding the oligonucleotide of some embodiments of the invention.

According to an aspect of some embodiments of the present invention there is provided a pharmaceutical composition comprising the oligonucleotide of some embodiments of the invention, or nucleic acid construct of some embodiments of the invention, and a pharmaceutically acceptable carrier or diluent.

According to an aspect of some embodiments of the present invention there is provided an article of manufacture comprising the oligonucleotide of some embodiments of the invention, or nucleic acid construct of some embodiments of the invention.

According to an aspect of some embodiments of the present invention there is provided an article of manufacture comprising the oligonucleotide of some embodiments of the invention, or nucleic acid construct of some embodiments of the invention, and an antibiotic.

According to an aspect of some embodiments of the present invention there is provided a method of treating or preventing a bacterial infection in a subject in need thereof, the method comprising administering to the subject the oligonucleotide of some embodiments of the invention, or nucleic acid construct of some embodiments of the invention, thereby treating or preventing the bacterial infection in the subject.

According to an aspect of some embodiments of the present invention there is provided a method of treating or preventing a bacterial infection in a subject in need thereof, the method comprising administering to the subject the oligonucleotide of some embodiments of the invention, or nucleic acid construct of some embodiments of the invention, and an antibiotic, thereby treating or preventing the bacterial infection in the subject.

According to an aspect of some embodiments of the present invention there is provided an oligonucleotide of some embodiments of the invention, or nucleic acid construct of some embodiments of the invention, for use in treating or preventing a bacterial infection in a subject in need thereof.

According to an aspect of some embodiments of the present invention there is provided an oligonucleotide of some embodiments of the invention, or nucleic acid construct of some embodiments of the invention, and an antibiotic for use in treating or preventing a bacterial infection in a subject in need thereof.

According to an aspect of some embodiments of the present invention there is provided a surface coated with the oligonucleotide of some embodiments of the invention, or nucleic acid construct of some embodiments of the invention.

According to an aspect of some embodiments of the present invention there is provided a surface coated with the oligonucleotide of some embodiments of the invention, or nucleic acid construct of some embodiments of the invention, and an antibiotic.

According to some embodiments of the invention, the ESBL is selected from the group consisting of:
SHV: sulfhydryl variable: beta-lactamase: EC: 3.5.2.6,
TEM: extended spectrum beta-lactamase: EC: 3.5.2.6,
OXA: oxacillin hydrolyzing enzyme: EC: 3.5.2.6, and
CTX-M: beta-lactamase: EC: 3.5.2.6.

According to some embodiments of the invention, the Carbapenemase is KPC: Carbapenem-hydrolyzing beta-lactamase, EC: 3.5.2.6.

According to some embodiments of the invention, the at least one DNAzyme comprises a plurality of DNAzymes.

According to some embodiments of the invention, the nucleic acid construct comprises a cleavable sequence between each pair of the plurality of DNAzymes.

According to some embodiments of the invention, the nucleic acid construct comprises a promoter sequence between each pair of the plurality of DNAzymes.

According to some embodiments of the invention, the DNAzyme molecule comprises no more than 70 nucleotides.

According to some embodiments of the invention, the DNAzyme molecule comprises a catalytic core of no more than 50 nucleotides.

According to some embodiments of the invention, the nucleic acid sequence comprises at least one modification.

According to some embodiments of the invention, the modification is in a catalytic core of the DNAzyme molecule.

According to some embodiments of the invention, the modification is in a binding arm of the DNAzyme molecule.

According to some embodiments of the invention, the modification comprises an insertion, a deletion, a substitution or a point mutation of at least one nucleic acid.

According to some embodiments of the invention, the modification comprises a modification that increases the stability or prevents degradation of the DNAzyme.

According to some embodiments of the invention, the modification comprises the addition of one or more nucleotide on a 5′ and/or 3′ terminus of the nucleic acid sequence.

According to some embodiments of the invention, the modification is selected from the group consisting of base modification, a sugar modification and an internucleotide linkage modification.

According to some embodiments of the invention, the modification is selected from the group consisting of locked nucleic acids (LNA), phosphorothioate, 2-O-fluor, 2-O-methyl, 2-O-methoxyethyl, methylcytosine, 2-fluoro and a 2-Fluoroarabinooligonucleotides.

According to some embodiments of the invention, the nucleic acid sequence is as set forth in any one of SEQ ID NOs: 1-26 or 35-53.

According to some embodiments of the invention, the oligonucleotide of some embodiments of the invention being at least 90% identical to the oligonucleotide sequence set forth in any one of SEQ ID NOs: 1-26 or 35-53.

According to some embodiments of the invention, the oligonucleotide of some embodiments of the invention being at least 80% identical to the oligonucleotide sequence set forth in any one of SEQ ID NOs: 1-26 or 35-53.

According to some embodiments of the invention, the oligonucleotide of some embodiments of the invention being at least 70% identical to the oligonucleotide sequence set forth in any one of SEQ ID NOs: 1-26 or 35-53.

According to some embodiments of the invention, the oligonucleotide of some embodiments of the invention being at least 60% identical to the oligonucleotide sequence set forth in any one of SEQ ID NOs: 1-26 or 35-53.

According to some embodiments of the invention, the DNAzyme is a 10-23 type DNAzyme molecule.

According to some embodiments of the invention, the DNAzyme is a 8-17 type DNAzyme molecule.

According to some embodiments of the invention, the oligonucleotide or nucleic acid construct of some embodiments of the invention is formulated with or attached to a permeability enhancing moiety.

According to some embodiments of the invention, the permeability enhancing moiety is a cholesterol moiety, a cell penetrating peptide, a lipid nanoparticle or a viral capsid.

According to some embodiments of the invention, the bacteria is a Gram positive bacteria.

According to some embodiments of the invention, the bacteria is a Gram negative bacteria.

According to some embodiments of the invention, the bacteria is selected from the group consisting of a Enterococcus faecium, a Staphylococcus aureus, a Klebsiella pneumoniae, an Acinetobacter baumannii, a Pseudomonas aeruginosa and an Enterobacter.

According to some embodiments of the invention, the bacteria is a Klebsiella pneumoniae.

According to some embodiments of the invention, the oligonucleotide or nucleic acid construct and the antibiotic are in a co-formulation.

According to some embodiments of the invention, the oligonucleotide or nucleic acid construct and the antibiotic are in separate formulations.

According to some embodiments of the invention, the subject is a human subject.

According to some embodiments of the invention, the subject is a non-human subject.

According to some embodiments of the invention, the antibiotic is a β-lactam.

According to some embodiments of the invention, the antibiotic is a carbapenem.

According to some embodiments of the invention, the antibiotic is a penicillin.

According to some embodiments of the invention, the antibiotic is a cephalosporin.

According to some embodiments of the invention, the antibiotic is a monobactam.

According to some embodiments of the invention, the antibiotic is selected from the group consisting of penicillin, methicillin, oxacillin, cephalosporin, aztreonam, cefoxitin, carbapenem, imipenem and meropenem.

According to some embodiments of the invention, the oligonucleotide, the nucleic acid construct, the pharmaceutical composition, the article of manufacture, the method, the oligonucleotide or nucleic acid construct for use and/or the surface of some embodiments of the invention, comprises or consists of ribonucleotides, deoxyribonucleotides or combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A illustrates a schematic illustration of the binding of 10-23 DNAzyme to its target RNA.

FIG. 1B illustrates the conservation of the target region of the DNAzyme KPC-337 targeting the bla carbapenemase in clinical isolates of Klebsiella pneumoniae (ec number 3.5.2.6, uniport ID Q9F663).

FIG. 2A illustrates entry of cholesterol-modified DNAzymes into bacteria. Specifically, the entry of DNAzymes into S. aureus and P. aeruginosa was observed when the DNAzyme was conjugated to cholesterol-TEG at the 3′, as shown by flow cytometry. Average fluorescent intensity of the bacterial population after incubation with the fluorescent DNAzyme.

FIG. 2B illustrates a screening of DNAzymes against various antibiotic resistance genes in Klebsiella pneumoniae.

FIG. 2C illustrates the MIC (minimal inhibitory concentration) test indicating the concentration of the antibiotic compound meropenem used in the screen of FIG. 2B.

FIG. 2D illustrates the selection of effective DNAzymes against various antibiotic resistance genes in Klebsiella pneumoniae from the screening provided in FIG. 2B.

FIGS. 3A-B illustrate uptake of DNAzymes into Klebsiella pneumoniae. Uptake of fluorescent DNAzyme, in media mimicking the in vivo conditions in the presence of subtoxic concentrations of meropenem as judged by flow cytometry. (FIG. 3A) Average fluorescent intensity and (FIG. 3B) histogram of the bacterial population after 4 hours of incubation with the fluorescent DNAzyme. Black line represent the basal fluorescence levels of the bacteria, dark grey represent fluorescent DNAzyme without modification, light grey line represent fluorescent DNAzyme with cholesterol-TEG modification at the 3′ end.

FIGS. 4A-B illustrate the bioactivity of DNAzyme KPC-337. (FIG. 4A) addition of KPC-337 to bacterial culture of Klebsiella pneumoniae reduced the transcript levels of bla-KPC; (FIG. 4B) a reduction of protein quantities is exemplified by the significant reduction in the β-lactamase activity of the strain performed with a colorimetric assay.

FIGS. 5A-C illustrate the sensitization of resistant Klebsiella pneumoniae by DNAzyme (KPC-337). (FIG. 5A) shows by optical density the relative growth of the bacteria in given antibiotic concentrations, and the significant reduction of the MIC (minimal inhibitory concentration) in resistant bacterial strain exposed to the DNAzyme KPC-337; (FIG. 5B) shows the growth inhibitory effect of the combination of subtoxic meropenem concentration and DNAzyme KPC-337 exemplified by the complete arrest of growth of strain; (FIG. 5C) the reduction of cell counts of KP culture is demonstrated as a function of time after the application of DNAzyme and meropenem, and indicates the bactericidal effect of the combination. All experiments in this figure include a control of a random non catalytic sequence designated “scramble”.

FIGS. 6A-B illustrate the bactericidal effect of a combination of subtoxic meropenem and KPC-337 DNAzyme on Klebsiella pneumoniae infection of a lung tissue. (FIG. 6A) demonstrates the significant reduction of growth in antibiotic resistant Klebsiella pneumoniae strain ATCC® BAA-1705™ cells infecting the tissue; (FIG. 6B) demonstrates a significant effect on free living cells that remained in the growth media. Of note, as expected, treatment in meropenem alone was not effective. All experiments in these figures included a control of a random non catalytic sequence designated “scramble”.

FIGS. 7A-B illustrate the beneficial effect of a combination of subtoxic effects of meropenem and DNAzyme KPC-337 in in vivo models. (FIG. 7A) shows a reduction of toxicity of Klebsiella pneumoniae strain ATCC® BAA-1705™ infection as judged by the viability of moth larvae; (FIG. 7B) shows the significant reduction of growth in bacterial cell count of antibiotic resistant Klebsiella pneumoniae strain ATCC® BAA-1705™ infecting a murine thigh. All experiments in these figures include a control of a random non catalytic sequence designated “scramble”.

FIG. 8 illustrates the lack of toxicity of DNAzyme KPC-337 to human HT-29 cells in bioactive concentrations (e.g. <5 μM).

FIGS. 9A-B illustrate the sensitization of methicillin-resistant Staphylococcus aureus (MRSA) by effective DNAzymes that target resistance genes. The figures show by optical density the growth curves of the bacteria in the presence of sub-toxic antibiotic concentration. Of note, treatment with DNAzymes delays the growth of the bacteria.

FIG. 10 illustrates a dose response assay for a specific DNAzyme that targets the resistant gene mecA in MRSA. The figure shows by optical density the growth curve of the bacteria in the presence of increasing concentrations of the DNAzyme mecA-658 (as set forth in SEQ ID NO: 20). Of note, increasing the concentration of the DNAzyme increases the growth delay of the bacteria.

FIG. 11 illustrates the effect of arm extension on DNAzymes against MRSA. Of note, optimization of the length of the arms of the DNAzymes increases the effectiveness of the DNAzymes in delaying the growth of the bacteria. The figure shows by optical density the growth curve of the bacteria in the presence of DNAzymes with optimized arms lengths, this leads to increase of the growth of the lag time of the bacteria (compared to FIG. 10).

FIG. 12 illustrates the selection of a highly effective DNAzyme against MRSA. The figure shows by optical density the growth curve of the bacteria in the presence of sub-optimal antibiotic concentration in the presence of effective DNAzyme FemA-545 (SEQ ID NO: 25) that targets the gene femA, this leads to inhibition of the bacterial growth for 44 hours.

### DESCRIPTION OF SPECIFIC EMBODIMENTS OF THE INVENTION

The present invention, in some embodiments thereof, relates to DNAzymes targeting bacterial target genes and, more particularly, but not exclusively, to the use of same for treating or preventing bacterial infections.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

ESKAPE pathogens, which include both Gram-positive and Gram-negative species, are a major challenge on healthcare due to their increased antibiotic resistance. In order to overcome antibiotic resistance, antibacterial antisense oligonucleotides that specifically inhibit essential gene expression and achieve gene-specific antibacterial effects have been described. However, these antisense oligonucleotides can only work on a single target and thus cannot completely reduce RNA transcript level in the cell. Furthermore, antisense molecules are sensitive to chemical and enzymatic degradation.

While reducing the present invention to practice, the present inventors have designed DNA enzymes (DNAzymes) which are cable of specifically targeting and cleaving RNA transcripts of selected bacterial antibiotic resistance genes, i.e. target genes which are highly conserved between bacteria, which are important for the resistance mechanism, which are transmittable between bacteria, and/or which are found in the clinic as the cause of infection. Administering these DNAzymes to bacteria is cytotoxic to the bacteria and renders the bacteria susceptible to antibiotics.

Specifically, the present inventors have designed and generated 45 DNAzymes capable of targeting various bacterial resistance genes including KPC, SHV-1, TEM, USA300HOU, mecA, mecR1, OXA-18, glpT and FemA (see Tables 1 and 2, and Example 1, herein below). One DNAzyme, termed KPC-337 (as set forth in SEQ ID NO: 1), was designed to target a conserved region in RNA transcripts of the gene bla carbapenemase of clinically relevant strains of Klebsiella pneumoniae (Example 2, herein below). DNAzyme KPC-337 was capable of entering the bacteria, binding to and specifically cleaving the intracellular RNA transcripts of bla carbapenemase (Example 2, herein below). Furthermore, the combined treatment of DNAzyme KPC-337 with the carbapenem-type antibiotic meropenem was bactericidal, thus illustrating that increased susceptibility of the bacteria to the antibiotic has been achieved (Example 2, herein below). The bactericidal effect of treatment with sub-toxic levels of meropenem with DNAzyme KPC-337 was further illustrated in an ex vivo model of lung disease (Example 3, herein below) and in in vivo infection larvae and murine models (Example 4, herein below). Importantly, the DNAzyme KPC-337 was not toxic to human cells in bioactive concentration (Example 4, herein below).

Furthermore, DNAzymes targeting the resistance genes mecA, mecR1, glpT, femA and USA300HOU_2333 (as set forth in SEQ ID NO: 5-26) were designed to target a conserved region in their corresponding RNA transcripts in MRSA (Example 6, herein below). These DNAzyme were all illustrated as capable of entering the bacteria. Furthermore, the combined use of each of these DNAzymes with the β-lactam type antibiotic cefoxitin increased the lag time of the bacteria. Thus, the use of each of these specifically selected DNAzymes increased susceptibility of the bacteria to the antibiotic treatment (Example 6, herein below).

Furthermore, in order to enhance the delivery of the DNAzymes into bacterial strains and the stability of the DNAzymes, DNAzymes are designed to comprise modifications on the oligonucleotide (e.g. the addition of oligos on the binding arms) and/or by utilizing lipid nanoparticles that can selectively enter only bacterial cells. These methods improve the specificity of delivery to the target bacteria and can further decrease the toxicity of treatment (Example 7, herein below).

Thus, according to one aspect of the present invention there is provided an oligonucleotide comprising a nucleic acid sequence of at least one DNAzyme, the DNAzyme being capable of silencing at least one target gene of a bacteria to thereby render the bacteria susceptible to antibiotic treatment.

The term “bacteria” as used herein generally refers to a genus of prokaryotic microorganisms scientifically classified as such. Most bacteria can be classified as Gram-positive bacteria or Gram-negative bacteria.

Gram-positive bacteria relate to bacteria bounded by only a single unit lipid membrane and contain a thick layer (20-80 nm) of peptidoglycan, which retains the crystal violet stain in a Gram staining technique. Exemplary Gram-positive bacteria include, but are not limited to, Actinomyces israelli, Bacillus species, Bacillus antracis, Brevibacillus, Clostridium, Clostridium perfringens, Clostridium tetani, Cornyebacterium, Corynebacterium diphtheriae, Enterococcus (e.g. Enterococcus faecium), Erysipelothrix rhusiopathiae, Lactobacillus, Listeria, Mycobacterium, Staphylococcus (e.g. Staphylococcus aureus), Streptomyces and Streptococcus.

Gram-negative bacteria relate to bacteria bounded by a cytoplasmic membrane as well as an outer cell membrane, containing only a thin layer of peptidoglycan between the two membranes, which is unable to retain crystal violet stain in a Gram staining technique. Exemplary Gram-negative bacteria include, but are not limited to, Aerobacter, Aeromonas, Acinetobacter (e.g. Acinetobacter baumannii), Agrobacterium, Bacteroides, Bartonella, Bordetella, Borrelia, Brucella, Burkholderia, Campylobacter, Calymmatobacterium, Campylobacter, Capnocytophaga, Cardiobacterium, Citrobacter, Chlamydia, Chlamydophila, Eikenella, Enterobacter, Enterobacter aerogenes, Escherichia, Flavobacterium, Francisella, Fusobacterium, Fusobacterium nucleatum, Gardnerella, Haemophilus, Hafnia, Helicobacter, Kingella, Klebsiella (e.g. Klebsiella pneumoniae), Legionella, Leptospira, Morganella, Moraxella, Mycoplasma, Neisseria, Pasteurella (e.g. Pasteurella multocida), Plesiomonas, Prevotella, Proteus, Providencia, Pseudomonas (e.g. Pseudomonas aeruginosa), Porphyromonas, Rickettsia, Salmonella, Serratia, Shigella, Stenotrophomonas, Streptobacillus, Streptobacillus moniliformis, Stenotrophomonas, Spirillum, Treponema (e.g. Treponema pallidium, Treponema pertenue), Xanthomonas, Veillonella, Vibrio, and Yersinia.

Additional bacterial species, e.g. which are neither gram-positive or gram-negative, include, but are not limited to, Borelia.

According to one embodiment, the bacteria are pathogenic bacteria.

According to one embodiment, the bacteria cause a nosocomial infection.

According to one embodiment, the bacteria are resistant to an antimicrobial treatment, such as to an antibiotic. Exemplary antibiotics include, but are not limited to, β-lactam antibiotics such as penicillin, methicillin, oxacillin, cephalosporin (e.g., third-generation oxyimino-cephalosporins e.g., ceftazidime, cefotaxime, and ceftriaxone; or methoxy-cephalosporins, e.g., cephamycin and carbapenem), cefoxitin, cefamandole, cefoperazone, imipenem, meropenem, aztreonam; macrolide antibiotics such as erythromycin, erythromycin thiocyanate; aminoglycoside antibiotics such as streptomycin, kanamycin, neomycin; tetracycline antibiotics such as minocycline, doxycycline; fluoroquinolone antibiotics such as ciprofloxacin, gemifloxacin, levofloxacin, moxifloxacin, ofloxacin; and polypeptide antibiotics such as vancomycin. According to one embodiment, the bacteria are resistant to multiple antimicrobial treatments (i.e. multidrug resistant (MDR)).

According to a specific embodiment, the bacteria is an Enterococcus faecium, a Staphylococcus aureus, a Klebsiella pneumoniae, an Acinetobacter baumannii, a Pseudomonas aeruginosa or an Enterobacter.

According to a specific embodiment, the bacteria is Klebsiella pneumoniae (also referred to as K. pneumoniae).

According to a specific embodiment, the bacteria is Staphylococcus aureus.

According to a specific embodiment, the bacteria is methicillin-resistant Staphylococcus aureus (MRSA).

According to a specific embodiment, the bacteria is Pseudomonas aeruginosa.

According to one embodiment, the phrase “render the bacteria susceptible to antibiotic treatment” refers to increasing susceptibility of the bacteria such that the bacteria are more susceptible to an antibiotic treatment by at least about 10%, 20%, 30%, 40%, 50%, 60%, 70%, 80%, 90% or 100% as compared to bacteria not treated by the at least one DNAzyme of the invention.

Bacteria which are more susceptible to treatment will typically exhibit suspension of growth and cell death, i.e. bactericidal effect, upon treatment with antibiotics. Methods for determining growth or death of bacteria are well known in the art. By way of example, the quantity of a target bacterial species or strain can be determined by growth in a culture, such as a liquid culture. In this regard, as the bacteria multiply and increase in number, the optical density of the liquid culture increases (due to the presence of an increasing number of bacterial cells). Thus, an increase in optical density indicates bacterial growth while a decrease in optical density indicates a decline in bacterial growth and bacterial death. For example, optical density (at for example at 600 nm) can be determined within the wells of a multi-well plate (e.g. a 96-well plate) using an automated plate reader.

According to one embodiment, the target gene of a bacteria is a resistance gene.

As used herein, the term “resistance gene” or “antibiotic resistance gene” can interchangeably be used to refer to a gene conferring antibiotic resistance in bacteria.

The resistance gene of some embodiments of the invention can be either a genomic or a plasmid gene, or from other sources such as genetic vectors acquired or engineered into the bacteria.

The bacterial target gene of some embodiments of the invention is the RNA transcript of a bacterial resistance gene (e.g. target RNA, as further discussed below).

The antibiotics targeted by the resistance gene can be, for example, β-lactams, macrolides, aminoglycosides, tetracyclines, fluoroquinolones and polypeptide antibiotics (as discussed in detail above).

Various resistance genes conferring resistance to antibiotics have been identified.

According to one embodiment, the resistance gene confers β-lactam antibiotic resistance.

According to one embodiment, the resistance gene confers carbapenem antibiotic resistance.

According to a specific embodiment, the resistance gene confers penicillin antibiotic resistance.

According to a specific embodiment, the resistance gene confers cephalosporin antibiotic resistance.

According to a specific embodiment, the resistance gene confers monobactam antibiotic resistance.

Target genes according to some embodiments of the invention include, but are not limited to, extended-spectrum beta-lactamases (ESBLs), penicillinases (EC: 3.5.2.6), cephalosporinases (EC: 3.5.2.6), and carbapenemases (EC: 3.5.2.6).

According to one embodiment, ESBLs includes, but are not limited to, TEM beta-lactamases (EC: 3.5.2.6), SHV beta-lactamases (EC: 3.5.2.6), CTX-M beta-lactamases (EC: 3.5.2.6), OXA beta-lactamases (EC: 3.5.2.6), PER beta-lactamases, VEB beta-lactamases, GES beta-lactamases, and IBC beta-lactamases.

Exemplary TEM beta-lactamases include, but are not limited to, TEM-1, TEM-2, TEM-3, TEM-4, TEM-10, TEM-12, TEM-13, and TEM-26.

Exemplary SHV beta-lactamases include, but are not limited to, SHV-1, SHV-3, SHV-4, SHV-5, SHV-6, SHV-9, SHV-12. According to a specific embodiment, SHV beta-lactamase comprises SHV-1. Additional SHV beta-lactamases which can be used according to some embodiments of the invention are discussed in Liakopoulos et al. Front. Microbiol., (2016), incorporated herein by reference.

Exemplary OXA beta-lactamases include, but are not limited to, OXA-1, OXA-2, OXA-10, OXA-11, OXA-14, OXA-15, OXA-16, OXA-18, OXA-23, OXA-51, OXA-58. According to a specific embodiment, OXA beta-lactamase comprises OXA-18.

Exemplary CTX-M beta-lactamases include, but are not limited to, CTX-M-15, CTX-M-14, CTX-M-3, and CTX-M-2.

According to one embodiment, penicillinases include, but are not limited to, PC1.

According to one embodiment, cephalosporinases include, but are not limited to, CepA, ACT-1, FOX-1, MIR-1, CMY.

According to one embodiment, carbapenemases include, but are not limited to, KPC (Klebsiella pneumoniae carbapenemase), OXA (oxacillinase) β-lactamases, VIM (Verona integron-encoded metallo-β-lactamase) and IMP-type carbapenemases (metallo-β-lactamases).

Exemplary KPC include, but are not limited to, KPC-1, KPC-2, KPC-3, KPC-4, KPC-5, KPC-6, KPC-7, KPC-8, KPC-9, KPC-10 or KPC-11. According to a specific embodiment, KPC comprises KPC-2.

A list of antibiotic agents of resistance, target genes and their corresponding ESKAPE pathogens is provided in Table 3 below, incorporated and modified from Santajit et al. Biomed Res Int. (2016): 2475067:

TABLE 3

| Antimicrobial | | Description of the | |
|---|---|---|---|
| agents | Target genes | target genes | ESKAPE pathogens |
| Narrow and | ACT-1, FOX-1, | Cephalosporinases | Enterobacter spp. |
| extended- | MIR-1, CMY | | |
| spectrum | | | |
| cephalosporins | | | |
| Penicillins | PC1 | Penicillinases | Enterobacteriaceae |
| Penicillins, | TEM-1, TEM-2, | Extended broad- | (e.g., K. pneumoniae, |
| cephalothin | TEM-13, SHV-1, | spectrum enzymes | Enterobacter spp.) |
| | SHV-11 | (ESBLs) | and nonfermenters |
| Penicillins, | TEM-3, TEM-10, | Extended broad- | (i.e., P. aeruginosa, |
| oxyimino- | TEM-26, SHV-2, | spectrum enzymes | A. baumannii) |
| cephalosporins | SHV-3, Klebsiella | (ESBLs) | |
| (cefotaxime, | oxytoca K1, CTX- | | |
| ceftazidime, | M, PER, VEB | | |
| ceftriaxone, | | | |
| cefepime), | | | |
| monobactams | | | |
| Penicillins, | TEM-30, TEM-31, | Extended broad- | |
| resistant to | SHV-10, SHV-72 | spectrum enzymes | |
| clavulanic acid, | | (ESBLs) with reduced | |
| tazobactam, | | binding to clavulanic | |
| sulbactam | | acid | |
| Penicillins, | TEM-50, TEM-158 | Extended-spectrum | |
| oxyimino- | | enzymes | |
| cephalosporins, | | | |
| monobactams, | | | |
| resistant to | | | |
| clavulanic acid, | | | |
| tazobactam, | | | |
| sulbactam | | | |
| Penicillins, | PSE-1, CARB-3 | Carbenicillin- | |
| carbenicillin | | hydrolyzing enzymes | |
| Carbenicillin, | RTG-4 (CARB-10) | Extended-spectrum | |
| cefepime | | carbenicillinase | |
| Cloxacillin, | OXA-1, OXA-2, | Cloxacillin-hydrolyzing | |
| oxacillin | OXA-10 | enzymes | |
| Cloxacillin, | OXA-11, OXA-15 | | |
| oxacillin, | | | |
| oxyimino- | | | |
| cephalosporins, | | | |
| monobactams | | | |
| Cloxacillin, | OXA-23, OXA-51, | | |
| oxacillin, | OXA-58 | | |
| carbapenems | | | |
| Cephalosporins | CepA | Cephalosporinases | |
| All β-lactams, | KPC, SME, GES, | Carbapenem- | |
| including | IMI-1 | hydrolyzing non- | |
| carbapenems | | metallo-β-lactamases | |
| All β-lactams, | IMP, VIM, IND | Metallo-β-lactamases | |
| including | | | |
| carbapenems, | | | |
| with exception | | | |
| of monobactams | | | |
| Penicillins | Penicillinases | Penicillinases | |
| | from Burkholderia | | |
| | cepacia | | |

Target genes according to specific embodiments of the invention include, but are not limited to:
Carbapenem-resistant Klebsiella pneumoniae www(dot)genomes(dot)atcc(dot)org/genomes/5d2426f33718400d?_ga=2(dot)196459244(dot)11 74834277(dot)1604822679-1567015497(dot)1603098978
Carbapenemase: (5R)-carbapenem-3-carboxylate synthase: EC: 1.14.20.3 (e.g. Gene symbol: carC, e.g. UniProtKB No. Q9XB59), e.g. KPC: Carbapenem-hydrolyzing beta-lactamase EC: 3.5.2.6 (e.g. bla-KPC2, Gene symbol: bla, e.g. UniProtKB No. Q9F663),
SHV-1: beta-lactamase: EC: 3.5.2.6 (e.g. Gene symbol: bla, e.g. UniProtKB No. POAD63)
TEM: extended spectrum beta-lactamase: EC: 3.5.2.6 (e.g. Gene symbol: bla, e.g. UniProtKB No. P62593)
OXA: oxacillin hydrolyzing enzyme: EC: 3.5.2.6 (e.g. Gene symbol: bla, e.g. UniProtKB No. 007293)
CTX-M: beta-lactamase: EC: 3.5.2.6 (e.g. Gene symbol: bla, e.g. UniProtKB No. P74841) Methicillin-resistant Staphylococcus aureus (MRSA) www(dot)genomes(dot)atcc(dot)org/genomes/b7dee0927a0b48c0?tab=overview-tab USA300HOU
mecA: penicillin-binding protein 2A (Gene symbol: mecA, e.g. UniProtKB No. P37958) mecR1: Beta-lactamase-sensing transmembrane signaling protein (Gene symbol: mecR1, e.g. UniProtKB No. POAOBO)
mecR2: MecR2 Methicillin resistance anti-repressor (Gene symbol: mecR2, e.g. UniProtKB No. U6EFM8)
glpT: glycerol-3-phosphate transporter (Gene symbol: glpT, e.g. UniProtKB No. P08194) femA: aminoacyltransferase (Gene symbol: femA, e.g. UniProtKB No. Q2FYR2)

According to one embodiment, when the bacteria are resistant to multiple antimicrobial treatments, such as in cases of mixed infection, one or more DNAzymes can be used for different targets.

The term “nucleic acid” as used herein generally refers to a molecule (single-stranded or double-stranded oligomer or polymer) of DNA or RNA or a derivative, mimic or analog thereof, comprising a nucleobase. A nucleobase includes, for example, a naturally occurring purine or pyrimidine base found in DNA (e.g., an adenine “A”, a guanine “G”, a thymine “T”, or a cytosine “C”) or a naturally occurring purine or pyrimidine base found in RNA (e.g., an adenine “A”, a guanine “G”, an uracil “U” or a cytosine “C”). The term “nucleic acid” encompasses the terms “oligonucleotide” and “polynucleotide” and “nucleotides”, each as a subgenus of the term “nucleic acid”. The term “nucleic acid” further includes nucleic acids derived from synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to respective naturally occurring portions, as further discussed herein below.

The term “oligonucleotide” as used herein refers to a short single stranded or double stranded sequence of nucleic acid such as ribonucleic acid (RNA), deoxyribonucleic acid (DNA), analogues or mimetics thereof. The term encompasses chemical modifications to the DNA and RNA. Without being limited thereto, the nucleic acid has typically less than or equal to 150 nucleotides.

The terms “nucleotide base” and “nucleotide” and “nucleic acid base” are used herein interchangeably and refer to a DNA or RNA base and any modification thereof.

The term “DNAzyme” (also referred to as a “DNA enzyme” or “deoxyribozyme”) as used herein refers to a DNA molecule that has complementarity in a substrate binding domain or region to a ribonucleic acid (RNA) substrate and is capable of catalyzing a modification (such as a cleavage) of the nucleic acid substrate. Typically, the complementarity functions to allow sufficient hybridization of the DNAzyme molecule to the substrate at a target region to allow the intermolecular cleavage of the substrate to occur thereby functionally inactivating it. The cleavage can occur via two optional mechanisms: (i) intrinsic catalytic activity of the DNAzyme (ii) recruitment of RNAse H to the hybridization site, leading to cleavage of the target RNA.

Thus, the term “mediating cleavage” refers to direct catalytic activity (i.e. by the DNAzyme molecule) or indirect catalytic activity (i.e. by an enzyme recruited by the DNAzyme molecule) which leads to cleavage and RNA interference of the resistance gene mRNA.

The term “target RNA” or “target region of an RNA” refers to an RNA molecule (e.g. an mRNA molecule encoding a resistance gene product) that is a target for downregulation. Similarly, the phrase “target site” refers to a sequence within a target RNA (e.g. within the antibiotic resistance mRNA sequence) that is “targeted” for cleavage mediated by the DNAzyme molecule that contains sequences within its substrate binding domains that are complementary to the target site (as discussed below).

According to one embodiment, silencing of the antibiotic resistance mRNA (e.g. cleavage of the antibiotic resistance mRNA) results in downregulation of mRNA and/or protein expression levels.

According to one embodiment, down regulating antibiotic resistance gene expression level refers to the absence of resistance gene mRNA and/or protein, as detected by RT-PCR or Western blot/Immunofluorescent staining, respectively.

The reduction may be by at least a 10%, at least 20%, at least 30%, at least 40%, at least 50%, at least 60%, at least 70%, at least 80%, at least 90%, at least 95% or at least 99% reduction, as compared to the expression level of resistance gene prior to the treatment.

Down regulation of expression may be either transient or permanent.

According to one embodiment, the DNAzyme molecule is synthetic. As used herein “synthetic” refers to a non-natural molecule.

The DNAzyme typically comprises a pair of binding arms which are complementary to binding regions on the nucleic acid substrate (e.g. resistant gene mRNA). Each binding arm of the DNAzyme comprises a number of nucleotides to permit sufficient bonding between the DNAzyme and its substrate to facilitate DNAzyme activity (i.e. cleavage of the resistant gene substrate at the target cleavage site). The binding arms may be the same or different lengths. Furthermore, the binding arms may comprise modified nucleotides, including modified bases, backbone, sugars and/or linkages to the extent that such modifications do not have an adverse effect on binding activity of the DNAzyme to the substrate (e.g. resistant gene mRNA). Such modifications are discussed below.

For example, each binding arm may comprise 5-25 nucleotides, 5-10 nucleotides, 10-15 nucleotides, 15-20 nucleotides or 20-25 nucleotides (e.g. deoxyribonucleotides or ribonucleotides). According to one embodiment, each binding arm comprises at least about 5, 6, 7, 8, 9, 10, 11, 12, 13, 14, 15, 16, 17, 18, 19, 20, 21, 22, 23, 24 or 25 nucleotides (e.g. deoxyribonucleotides or ribonucleotides). According to a specific embodiment, each binding arm comprises at least about 8-10 nucleotides, e.g. 9 nucleotides (e.g. deoxyribonucleotides or ribonucleotides).

According to a specific embodiment, each of the binding arms of the DNAzyme molecule comprises no more than 6, 7, 8, 9, 10, 11, 12, 13, 14 or 15 nucleotides (e.g. deoxyribonucleotides or ribonucleotides).

As mentioned, the DNAzyme targets a nucleic acid-based substrate comprising binding regions which are essentially complementary to the binding arms of the DNAzyme, and which hybridize with the binding arms of the DNAzyme. The binding regions need not be fully complementary with the binding arms of the DNAzyme, provided that they hybridize sufficiently with the DNAzyme such that the catalytic activity of the DNAzyme is not adversely affected (e.g. exhibit at least about 70%, 80%, 85%, 90%, 95%, 97% or 99% complementarity). Typically, the cleavage site is within a target region of the substrate situated between the binding regions. The terminal 5′- and 3′ ends of the target region are each linked to a binding region at the appropriate corresponding terminus (e.g. 5′ to 3′) of the binding arm.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, binding with specificity by substrate binding domains of a DNAzyme of some embodiments of the invention such that the catalytic domain of the DNAzyme is brought in to close enough proximity with a target sequence to permit catalytic cleavage of the target sequence. The degree of complementarity between the substrate binding domains of the DNAzyme and the target region of a RNA (e.g. a resistant gene mRNA) can vary, but no more than by what is required in order to permit the DNAzyme to cleave or mediate cleavage (e.g. by RNase H) of the target region. Determination of binding free energies for nucleic acid molecules to determine percent complementarity is well known in the art. See e.g., Freier et al., 1986; Turner et al., 1987.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). The terms “100% complementary”, “fully complementary”, and “perfectly complementary” indicate that all of the contiguous residues of a nucleic acid sequence can hydrogen bond with the same number of contiguous residues in a second nucleic acid sequence.

The DNAzyme additionally comprises a catalytic domain (also referred to as catalytic core) between the binding arms, generally in the form of a loop, which includes single-stranded DNA, and may optionally include double-stranded regions. The terminal 5′- and 3′ ends of the catalytic domain are each linked to a binding arm at the appropriate corresponding terminus of the binding arm (e.g. 5′ to 3′). The catalytic region may incorporate modified nucleotides, including modified bases, backbone, sugars and/or linkages to the extent that such modifications do not have an adverse effect on catalytic activity (i.e. cleavage activity) of the DNAzyme, such modifications are discussed below.

The size of the catalytic domain in each DNAzyme may include, for example, 5-100 nucleotides, e.g. 5-10 nucleotides, 10-20 nucleotides, 20-30 nucleotides, 30-40 nucleotides, 40-50 nucleotides, 50-75 nucleotides or 75-100 nucleotides (e.g. deoxyribonucleotides or ribonucleotides). According to one embodiment, the catalytic domain comprises at least about 5, 10, 15, 20, 25, 30, 35, 40, 45 or 50 nucleotides (e.g. deoxyribonucleotides or ribonucleotides). According to a specific embodiment, the catalytic domain comprises at least about 10, 11, 12, 13, 14, 15, 16, 17, 18, 19, 20, 21, 22, 23, 24, 25, 30, 35, 40, 45 or 50 nucleotides (e.g. deoxyribonucleotides or ribonucleotides).

According to a specific embodiment, the catalytic domain of the DNAzyme molecule comprises no more than 14, 15, 16, 17, 18, 19, 20, 21, 22, 23, 24, 25, 30, 35, 40, 45 or 50 nucleotides (e.g. deoxyribonucleotides or ribonucleotides).

Exemplary DNAzyme catalytic domains which may be used in accordance with some embodiments of the invention, include, but are not limited to, the catalytic cores of lanthanide-dependent DNAzymes such as Ce13d, Lu12 and Tm7; the catalytic cores of magnesium-dependent DNAzymes such as 17E and 10-23, 8-17; the catalytic cores of uranyl-specific DNAzymes such as 39E and EHg0T; the catalytic cores of lead-dependent DNAzymes such as GR5; and functionally equivalent DNAzyme catalytic cores derived from any of these which exhibit a high degree of sequence identity, e.g. at least about 80%, 85%, 90%, 95% or 99%. The term “functionally equivalent” refers to DNAzyme catalytic cores which retain the ability to cleave the DNAzyme substrate or to recruit RNase H. Additional catalytic cores of DNAzymes which may be used in accordance with some embodiments of the invention include e.g. Bipartite I and Bipartite II (discussed in Feldman and Sen. Journal of molecular biology (2001) 313(2): 283-294, incorporated herein by reference), 10MD5 and I-R3 (discussed in Hollenstein, Molecules (2015) 20(11): 20777-20804, and in Zhou et al. Theranostics. 2017; 7(4): 1010-1025, both incorporated herein by reference).

According to one embodiment, the DNAzyme is a 10-23 type DNAzyme (i.e. comprises the 10-23 catalytic core).

The term “10-23” refers to a general DNAzyme model (discussed in detail in Sontoro and Joyce, PNAS (1997) 94 (9):4262-4266, incorporated herein by reference). DNAzymes of the 10-23 model typically have a catalytic domain of 15 nucleotides, which are flanked by two substrate binding domains. The catalytic domain of 10-23 DNAzymes typically comprises the sequence ggctagctacaacga (SEQ ID NO: 28). The DNAzyme 10-23 typically cleaves mRNA strands that contain an unpaired purine-pyrimidine pair. The length of the substrate binding domains of 10-23 DNAzymes is variable and may be of either equal length or variable length. According to one embodiment, the length of the substrate binding domains ranges between 6 and 14 nucleotides, e.g. between 8 and 12 nucleotides (e.g. 7, 8, 9, 10, 11, 12 nucleotides, e.g. 9 nucleotides).

According to one embodiment, the DNAzyme is a 8-17 type DNAzyme (i.e. comprises the 8-17 catalytic core).

The term “8-17” refers to a general DNAzyme model (discussed in detail in Sontoro and Joyce, PNAS (1997) 94 (9):4262-4266, incorporated herein by reference). DNAzymes of the 8-17 model typically have a catalytic domain of 14 nucleotides, which are flanked by two substrate binding domains. The catalytic domain of 8-17 DNAzymes typically comprises the sequence TCCGAGCCGGACGA (SEQ ID NO: 29). The length of the substrate binding domains of 8-17 DNAzymes is variable and may be of either equal length or variable length. According to one embodiment, the length of the substrate binding domains ranges between 6 and 14 nucleotides, e.g. between 8 and 12 nucleotides (e.g. 7, 8, 9, 10, 11, 12 nucleotides, e.g. 8 nucleotides).

According to one embodiment, the DNAzyme catalytic domain comprises the 16.2-11 catalytic core: GTGACCCCUUG (SEQ ID NO: 30); the 9-86 catalytic core: UCAUGCAGCGCGUAGUGUC (SEQ ID NO: 31); the 12-91 catalytic core: UGAUGCAGCGCAUGUGUC (SEQ ID NO: 32); the FR17_6 catalytic core: AAGCAGUUAAGAC (SEQ ID NO: 33) or the Bipartite I or Bipartite II catalytic core: AAGGAGGTAGGGGTTCCGCTCC (SEQ ID NO: 34).

According to one embodiment, the DNAzyme is an inducible DNAzyme. Exemplary inducible DNAzymes are MNAzymes (discussed in detail in Mokany et al., Journal of the American Chemical Society (2010) 132(3): 1051-1059, incorporated herein by reference). Specifically, MNAzymes are multicomponent complexes that produce amplified “output” signals in response to specific “input” signals. Multiple oligonucleotide partzymes assemble into active MNAzymes only in the presence of an input assembly facilitator such as a target nucleic acid. Once formed, MNAzymes catalytically modify a generic substrate, generating an amplified output signal that heralds the presence of the target while leaving the target intact.

According to a specific embodiment, the DNAzyme molecule comprises 20-80 nucleotides, e.g. 20-70 nucleotides, e.g. 20-60 nucleotides, e.g. 20-50 nucleotides, e.g. 20-40 nucleotides, e.g. 20-30 nucleotides, e.g. 30-50 nucleotides, e.g. 30-40 nucleotides (e.g. deoxyribonucleotides or ribonucleotides). According to a specific embodiment, the DNAzyme molecule comprises at least about 20, 25, 30, 31, 32, 33, 34, 35, 36, 37, 38, 39, 40, 45, 50, 55, 60, 65, 70 or 75 nucleotides (e.g. deoxyribonucleotides or ribonucleotides).

According to a specific embodiment, the DNAzyme molecule comprises no more than 20-80 nucleotides, e.g. 20-70 nucleotides, e.g. 20-60 nucleotides, e.g. 20-50 nucleotides, e.g. 20-40 nucleotides, e.g. 20-30 nucleotides, e.g. 30-50 nucleotides, e.g. 30-40 nucleotides (e.g. deoxyribonucleotides or ribonucleotides). According to a specific embodiment, the DNAzyme molecule comprises no more than e.g. 30, 31, 32, 33, 34, 35, 36, 37, 38, 39, 40, 41, 42, 43, 44, 45, 50, 55, 60, 65, 70, 75 or 80 nucleotides (e.g. deoxyribonucleotides or ribonucleotides).

In some embodiments, one or more mutations or modifications within the catalytic domain of the DNAzyme molecule can be carried out to increase the catalytic activity of the DNAzyme molecule (e.g. by insertion, deletion, substitution or point mutation of nucleic acids). It is to be understood that any mutation or modification, e.g., within the substrate binding domain sequences or catalytic domain sequence, must not adversely impact the molecule's ability to induce catalysis (e.g. cleave) the specific substrate, i.e. the resistant gene RNA.

According to a specific embodiment, the modification comprises the insertion, deletion, substitution or point mutation of 1-10 nucleotides, e.g. 1-8 nucleotides, e.g. 1-6 nucleotides, e.g. 1-5 nucleotides, e.g. 1-4 nucleotides, e.g. 1-3 nucleotides, or e.g. 1-2 nucleotides in the catalytic domain of the DNAzyme.

According to a specific embodiment, the modification comprises the insertion, deletion, substitution or point mutation of 1, 2, 3, 4, 5, 6, 7, 8, 9, 10 nucleotides, e.g. 1 or 2 nucleotides, in the catalytic domain of the DNAzyme.

Additionally or alternatively, the DNAzyme molecule may be modified to alter the length of the substrate binding domains of the DNAzyme molecule. The substrate binding domains of the DNAzyme molecule have binding specificity for and associate with a complementary sequence of bases within a target region of a substrate nucleic acid sequence (as discussed above). Methods of altering the length of the recognition domains are known in the art and include direct synthesis and PCR, for example.

Alteration of the length of the recognition domains of a DNAzyme molecule can have a desirable effect on the binding specificity of the DNAzyme molecule. For example, an increase in the length of the substrate binding domains can increase binding specificity between the DNAzyme molecule and the complementary base sequences of a target region in a substrate polynucleotide (i.e. resistant gene RNA). In addition, an increase in the length of the substrate binding domains can also increase the affinity with which the DNA molecule binds to the polynucleotide substrate. In various embodiments, these altered substrate binding domains in the DNAzyme molecule confer increased binding specificity and affinity between the DNAzyme molecule and its substrate (i.e. resistant gene RNA), however, it may decrease catalytic efficiency of the DNAzyme. Therefore, one of skill in the art will appreciate that alteration of the length of the recognition domains is a balance of optimal binding and catalytic activity.

According to a specific embodiment, the modification comprises the addition of 1-10 nucleotides, e.g. 1-8 nucleotides, e.g. 1-6 nucleotides, e.g. 1-5 nucleotides, e.g. 1-4 nucleotides, e.g. 1-3 nucleotides, or e.g. 1-2 nucleotides to the substrate binding domain (i.e. binding arm) of the DNAzyme.

According to a specific embodiment, the modification comprises the addition of 1, 2, 3, 4, 5, 6, 7, 8, 9, 10 nucleotides, e.g. 1 or 2 nucleotides, to the substrate binding domain (i.e. binding arm) of the DNAzyme.

Additionally or alternatively, the substrate binding domains of the DNAzyme molecule may be modified to improve binding to the mRNA target (i.e. the RNA transcript of a bacterial resistance gene). Accordingly, the modification may comprise the insertion, deletion, substitution or point mutation of 1, 2, 3, 4, 5, 6, 7, 8, 9, 10 nucleotides, e.g. 1 or 2 nucleotides, in the substrate binding domain of the DNAzyme.

According to a specific embodiment, the DNAzyme molecule comprises a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth in any one of SEQ ID NOs: 1-26 or 35-53.

As used herein, “sequence identity” or “identity” in the context of two nucleic acid sequences includes reference to the residues in the two sequences which are the same when aligned.

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire nucleic acid sequences of the invention and not over portions thereof.

The degree of homology or identity between two or more sequences can be determined using various known sequence comparison tools. Following is a non-limiting description of such tools which can be used along with some embodiments of the invention.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used.

According to some embodiment, determination of the degree of homology further requires employing the Smith-Waterman algorithm (for protein-protein comparison or nucleotide-nucleotide comparison).

According to one embodiment, the DNAzyme molecule comprises a nucleic acid sequence at least 60% identical to the nucleic acid sequence set forth in any one of SEQ ID NOs: 1-26 or 35-53.

According to one embodiment, the DNAzyme molecule comprises a nucleic acid sequence at least 65% identical to the nucleic acid sequence set forth in any one of SEQ ID NOs: 1-26 or 35-53.

According to one embodiment, the DNAzyme molecule comprises a nucleic acid sequence at least 70% identical to the nucleic acid sequence set forth in any one of SEQ ID NOs: 1-26 or 35-53.

According to one embodiment, the DNAzyme molecule comprises a nucleic acid sequence at least 75% identical to the nucleic acid sequence set forth in any one of SEQ ID NOs: 1-26 or 35-53.

According to one embodiment, the DNAzyme molecule comprises a nucleic acid sequence at least 80% identical to the nucleic acid sequence set forth in any one of SEQ ID NOs: 1-26 or 35-53.

According to one embodiment, the DNAzyme molecule comprises a nucleic acid sequence at least 85% identical to the nucleic acid sequence set forth in any one of SEQ ID NOs: 1-26 or 35-53.

According to one embodiment, the DNAzyme molecule comprises a nucleic acid sequence at least 90% identical to the nucleic acid sequence set forth in any one of SEQ ID NOs: 1-26 or 35-53.

According to one embodiment, the DNAzyme molecule comprises a nucleic acid sequence at least 95% identical to the nucleic acid sequence set forth in any one of SEQ ID NOs: 1-26 or 35-53.

According to one embodiment, the DNAzyme molecule comprises a nucleic acid sequence at least 98% identical to the nucleic acid sequence set forth in any one of SEQ ID NOs: 1-26 or 35-53.

According to one embodiment, the DNAzyme molecule comprises a nucleic acid sequence at least 99% identical to the nucleic acid sequence set forth in any one of SEQ ID NOs: 1-26 or 35-53.

According to one embodiment, the DNAzyme molecule comprises a nucleic acid sequence set forth in any one of SEQ ID NOs: 1-26 or 35-53.

According to a specific embodiment, the DNAzyme molecule is DNAzyme KPC-337 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 1.

According to a specific embodiment, the DNAzyme molecule is DNAzyme KPC-337 comprising a nucleic acid sequence set forth in SEQ ID NO: 1.

According to a specific embodiment, the DNAzyme molecule is DNAzyme KPC-332 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 2 or 40.

According to a specific embodiment, the DNAzyme molecule is DNAzyme KPC-332 comprising a nucleic acid sequence set forth in SEQ ID NO: 2 or 40.

According to a specific embodiment, the DNAzyme molecule is DNAzyme SHV-1-133 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 3.

According to a specific embodiment, the DNAzyme molecule is DNAzyme SHV-1-133 comprising a nucleic acid sequence set forth in SEQ ID NO: 3.

According to a specific embodiment, the DNAzyme molecule is DNAzyme TEM-588 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 4.

According to a specific embodiment, the DNAzyme molecule is DNAzyme TEM-588 comprising a nucleic acid sequence set forth in SEQ ID NO: 4.

According to a specific embodiment, the DNAzyme molecule is DNAzyme USA300HOU-2333-1302 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 5, 18 or 19.

According to a specific embodiment, the DNAzyme molecule is DNAzyme USA300HOU-2333-1302 comprising a nucleic acid sequence set forth in SEQ ID NO: 5, 18 or 19.

According to a specific embodiment, the DNAzyme molecule is DNAzyme mecA-650 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 6, 13 or 14.

According to a specific embodiment, the DNAzyme molecule is DNAzyme mecA-650 comprising a nucleic acid sequence set forth in SEQ ID NO: 6, 13 or 14.

According to a specific embodiment, the DNAzyme molecule is DNAzyme mecA-661 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 7 or 15.

According to a specific embodiment, the DNAzyme molecule is DNAzyme mecA-661 comprising a nucleic acid sequence set forth in SEQ ID NO: 7 or 15.

According to a specific embodiment, the DNAzyme molecule is DNAzyme mecA-658 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 8, 20 or 26.

According to a specific embodiment, the DNAzyme molecule is DNAzyme mecA-658 comprising a nucleic acid sequence set forth in SEQ ID NO: 8, 20 or 26.

According to a specific embodiment, the DNAzyme molecule is DNAzyme USA300HOU-2396-437 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 9, 10 or 23.

According to a specific embodiment, the DNAzyme molecule is DNAzyme USA300HOU-2396-437 comprising a nucleic acid sequence set forth in SEQ ID NO: 9, 10 or 23.

According to a specific embodiment, the DNAzyme molecule is DNAzyme mecA-647 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 11 or 12.

According to a specific embodiment, the DNAzyme molecule is DNAzyme mecA-647 comprising a nucleic acid sequence set forth in SEQ ID NO: 11 or 12.

According to a specific embodiment, the DNAzyme molecule is DNAzyme glpT-1122 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 16 or 17.

According to a specific embodiment, the DNAzyme molecule is DNAzyme glpT-1122 comprising a nucleic acid sequence set forth in SEQ ID NO: 16 or 17.

According to a specific embodiment, the DNAzyme molecule is DNAzyme mecR1-146 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 21.

According to a specific embodiment, the DNAzyme molecule is DNAzyme mecR1-146 comprising a nucleic acid sequence set forth in SEQ ID NO: 21.

According to a specific embodiment, the DNAzyme molecule is DNAzyme mecA-353 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 22.

According to a specific embodiment, the DNAzyme molecule is DNAzyme mecA-353 comprising a nucleic acid sequence set forth in SEQ ID NO: 22.

According to a specific embodiment, the DNAzyme molecule is DNAzyme USA300HOU-2333-676 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 24.

According to a specific embodiment, the DNAzyme molecule is DNAzyme USA300HOU-2333-676 comprising a nucleic acid sequence set forth in SEQ ID NO: 24.

According to a specific embodiment, the DNAzyme molecule is DNAzyme femA-545 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 25.

According to a specific embodiment, the DNAzyme molecule is DNAzyme femA-545 comprising a nucleic acid sequence set forth in SEQ ID NO: 25.

According to a specific embodiment, the DNAzyme molecule is DNAzyme KPC-568 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 35.

According to a specific embodiment, the DNAzyme molecule is DNAzyme KPC-568 comprising a nucleic acid sequence set forth in SEQ ID NO: 35.

According to a specific embodiment, the DNAzyme molecule is DNAzyme KPC-36 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 36.

According to a specific embodiment, the DNAzyme molecule is DNAzyme KPC-36 comprising a nucleic acid sequence set forth in SEQ ID NO: 36.

According to a specific embodiment, the DNAzyme molecule is DNAzyme KPC-470 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 37.

According to a specific embodiment, the DNAzyme molecule is DNAzyme KPC-470 comprising a nucleic acid sequence set forth in SEQ ID NO: 37.

According to a specific embodiment, the DNAzyme molecule is DNAzyme KPC-389 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 38.

According to a specific embodiment, the DNAzyme molecule is DNAzyme KPC-389 comprising a nucleic acid sequence set forth in SEQ ID NO: 38.

According to a specific embodiment, the DNAzyme molecule is DNAzyme KPC-563 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 39.

According to a specific embodiment, the DNAzyme molecule is DNAzyme KPC-563 comprising a nucleic acid sequence set forth in SEQ ID NO: 39.

According to a specific embodiment, the DNAzyme molecule is DNAzyme KPC-574 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 41.

According to a specific embodiment, the DNAzyme molecule is DNAzyme KPC-574 comprising a nucleic acid sequence set forth in SEQ ID NO: 41.

According to a specific embodiment, the DNAzyme molecule is DNAzyme KPC-633 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 42.

According to a specific embodiment, the DNAzyme molecule is DNAzyme KPC-633 comprising a nucleic acid sequence set forth in SEQ ID NO: 42.

According to a specific embodiment, the DNAzyme molecule is DNAzyme KPC-344 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 43.

According to a specific embodiment, the DNAzyme molecule is DNAzyme KPC-344 comprising a nucleic acid sequence set forth in SEQ ID NO: 43.

According to a specific embodiment, the DNAzyme molecule is DNAzyme OXA-18-294 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 44.

According to a specific embodiment, the DNAzyme molecule is DNAzyme OXA-18-294 comprising a nucleic acid sequence set forth in SEQ ID NO: 44.

According to a specific embodiment, the DNAzyme molecule is DNAzyme OXA-18-59 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 45.

According to a specific embodiment, the DNAzyme molecule is DNAzyme OXA-18-59 comprising a nucleic acid sequence set forth in SEQ ID NO: 45.

According to a specific embodiment, the DNAzyme molecule is DNAzyme OXA-18-125 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%. 86%, 87%, 88%. 89%, 90%, 91%, 92%, 93%. 94%, 95%. 96%, 97%, 98%, 99% or 100% identical to the nucleic acid sequence set forth set forth in SEQ ID NO: 46.

According to a specific embodiment, the DNAzyme molecule is DNAzyme OXA-18-125 comprising a nucleic acid sequence set forth in SEQ ID NO: 46.

According to a specific embodiment, the DNAzyme molecule is DNAzyme OXA-18-225 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 47.

According to a specific embodiment, the DNAzyme molecule is DNAzyme OXA-18-225 comprising a nucleic acid sequence set forth in SEQ ID NO: 47.

According to a specific embodiment, the DNAzyme molecule is DNAzyme SHV-1-127 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 48.

According to a specific embodiment, the DNAzyme molecule is DNAzyme SHV-1-127 comprising a nucleic acid sequence set forth in SEQ ID NO: 48.

According to a specific embodiment, the DNAzyme molecule is DNAzyme SHV-1-33 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 49.

According to a specific embodiment, the DNAzyme molecule is DNAzyme SHV-1-33 comprising a nucleic acid sequence set forth in SEQ ID NO: 49.

According to a specific embodiment, the DNAzyme molecule is DNAzyme SHV-1-197 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 50.

According to a specific embodiment, the DNAzyme molecule is DNAzyme SHV-1-197 comprising a nucleic acid sequence set forth in SEQ ID NO: 50.

According to a specific embodiment, the DNAzyme molecule is DNAzyme TEM-518 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 51.

According to a specific embodiment, the DNAzyme molecule is DNAzyme TEM-518 comprising a nucleic acid sequence set forth in SEQ ID NO: 51.

According to a specific embodiment, the DNAzyme molecule is DNAzyme TEM-810 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 52.

According to a specific embodiment, the DNAzyme molecule is DNAzyme TEM-810 comprising a nucleic acid sequence set forth in SEQ ID NO: 52.

According to a specific embodiment, the DNAzyme molecule is DNAzyme TEM-14 comprising a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the nucleic acid sequence set forth set forth in SEQ ID NO: 53.

According to a specific embodiment, the DNAzyme molecule is DNAzyme TEM-14 comprising a nucleic acid sequence set forth in SEQ ID NO: 53. As mentioned, the DNAzyme molecules of some embodiments of the invention may be administered to the subject as a single DNAzyme molecule treatment. Alternatively, the DNAzyme molecules of some embodiments of the invention may be administered to the subject in combination (e.g. 2, 3, 4, 5 or more DNAzymes) in order to mediate cleavage of two or more target sites in resistance gene mRNA (e.g. in a single RNA target or in different RNA targets) thereby increasing efficiency of RNA silencing. Such a determination is well within the capability of one of skill in the art. Such DNAzymes may be utilized together, or subsequently to each other.

Various modifications to DNAzyme molecules can be made to enhance the utility of these molecules. Such modifications can enhance affinity for the nucleic acid target, increase activity, increase specificity, increase stability and decrease degradation (e.g. in the presence of nucleases), increase shelf-life, enhance half-life and/or improve introduction of such DNAzyme molecules to the target site (for example, to enhance penetration of cellular membranes, confer the ability to recognize and bind to targeted cells, and enhance cellular uptake), as discussed in further detail below.

According to one embodiment, the DNAzyme molecule comprises a modification selected from an insertion, deletion, substitution or point mutation of a nucleic acid, as long as the molecule retains at least about 80%, 85%, 90%, 95%, 99% or 100% of its the biological activity (i.e. silencing activity, e.g. mediating catalytic activity on the resistant gene substrate, i.e. the resistant gene RNA).

It will be appreciated that insertions, deletions, substitutions or point mutations can be generated using methods that produce random or specific alterations. These mutations or modifications can, for example, change the length of, or alter the nucleotide sequence of, a loop, a spacer region or the substrate binding domain (e.g. binding arm of the DNAzyme as discussed above) or add one or more non-nucleotide moieties to the molecule to, for example, increase stability and/or decrease degradation.

The DNAzyme molecule of the invention is optimally stable within cells until translation of the target RNA has been inhibited long enough to reduce the levels of the undesirable protein. This period of time varies between hours to days depending upon the cell type and state (e.g. type of bacteria). Although DNAzyme molecules as described herein are considered advantageous over RNA based molecules in that DNAzymes are less sensitive to degradation, in some embodiments it is desirable to further increase stability and nuclease resistance of the DNAzymes. Furthermore, when RNA based molecules are utilized, these may be modified to increase their stability and nuclease resistance.

According to one embodiment, the modification is selected from the group consisting of a nucleobase modification, a sugar modification, and an internucleotide linkage modification (e.g. phosphorus-modified internucleotide linkage), as is broadly described herein under.

According to one embodiment, the DNAzyme molecule comprises one or more chemical modifications.

Any chemical modification can be applied to the DNAzyme molecule of the invention as long as the molecule retains at least about 80%, 85%, 90%, 95%, 99% or 100% of its the biological activity (i.e. silencing activity, e.g. mediating catalytic activity on the resistant gene substrate, i.e. the resistant gene RNA).

According to one embodiment, the DNAzyme molecule includes at least one base (e.g. nucleobase) modification or substitution.

As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and 0-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications. Additional base modifications are described in Deleavey and Damha, Chemistry and Biology (2012) 19: 937-954, incorporated herein by reference.

According to one embodiment, the modification is in the backbone (i.e. in the internucleotide linkage and/or the sugar moiety).

Sugar modification of nucleic acid molecules have been extensively described in the art (see PCT International Publication Nos. WO 92/07065, WO 93/15187, WO 98/13526, and WO 97/26270; U.S. Pat. Nos. 5,334,711; 5,716,824; and U.S. Pat. No. 5,627,053; Perrault et al., 1990; Pieken et al., 1991; Usman & Cedergren, 1992; Beigelman et al., 1995; Karpeisky et al., 1998; Earnshaw & Gait, 1998; Verma & Eckstein, 1998; Burlina et al., 1997; all of which are incorporated herein by reference). Such publications describe general methods and strategies to determine the location of incorporation of sugar, base, and/or phosphate modifications and the like into nucleic acid molecules without modulating catalysis. Exemplary sugar modifications include, but are not limited to, 2′-modified nucleotide, e.g., a 2′-deoxy, 2′-fluoro (2′-F), 2′-deoxy-2′-fluoro, 2′-O-methyl (2′-O-Me), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-Fluoroarabinooligonucleotides (2′-F-ANA), 2′-O—N-methylacetamido (2′-O-NMA), 2′-NH2 or a locked nucleic acid (LNA). Additional sugar modifications are described in Deleavey and Damha, Chemistry and Biology (2012) 19: 937-954, incorporated herein by reference.

Thus, for example, oligonucleotides can be modified to enhance their stability and/or enhance biological activity by modification with nuclease resistant groups, for example, the DNAzyme molecule of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), e.g. inclusion of nucleic acid analogues in which the ribose ring is “locked” by a methylene bridge connecting the 2′-O atom and the 4′-C atom, ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to the target. The inclusion of pyranose sugars in the oligonucleotide backbone can also decrease endonucleolytic cleavage. The binding arms may further include peptide nucleic acid (PNA) in which the deoxribose (or ribose) phosphate backbone in the DNA is replaced with a polyamide backbone, or may include polymer backbones, cyclic backbones, or acyclic backbones. The binding regions may incorporate sugar mimetics, and may additionally include protective groups, particularly at terminal ends thereof, to prevent undesirable degradation (as discussed below).

Exemplary internucleotide linkage modifications include, but are not limited to, phosphorothioate, chiral phosphorothioate, phosphorodithioate, phosphotriester, aminoalkyl phosphotriester, methyl phosphonate, alkyl phosphonate (including 3′-alkylene phosphonates), chiral phosphonate, phosphinate, phosphoramidate (including 3′-amino phosphoramidate), aminoalkylphosphoramidate, thionophosphoramidate, thionoalkylphosphonate, thionoalkylphosphotriester, boranophosphate (such as that having normal 3′-5′ linkages, 2′-5′ linked analogues of these, and those having inverted polarity wherein the adjacent pairs of nucleoside units are linked 3′-5′ to 5′-3′ or 2′-5′ to 5′-2′), boron phosphonate, phosphodiester, phosphonoacetate (PACE), morpholino, amidate carbamate, carboxymethyl, acetamidate, polyamide, sulfonate, sulfonamide, sulfamate, formacetal, thioformacetal, alkylsilyl, substitutions, peptide nucleic acid (PNA) and/or threose nucleic acid (TNA). Various salts, mixed salts, and free acid forms of the above modifications can also be used. Additional internucleotide linkage modifications are described in Deleavey and Damha, Chemistry and Biology (2012) 19: 937-954; and Hunziker & Leumann, 1995 and De Mesmaeker et al., 1994, both incorporated herein by reference.

According to a specific embodiment, the modification comprises modified nucleoside triphosphates (dN*TPs).

According to one embodiment, the modification comprises an edge-blocker oligonucleotide.

According to a specific embodiment, the edge-blocker oligonucleotide comprises a phosphate, an inverted deoxythymidine (dT) and an amino-C7.

According to a specific embodiment, the modification comprises an inverted deoxythymidine (dT) positioned in at least one terminal end of the DNAzyme molecule. For example, an inverted dT can be incorporated at the 3′-end of an oligo, leading to a 3′-3′ linkage which inhibits both degradation by 3′ exonucleases and extension by DNA polymerases.

According to one embodiment, the DNAzyme molecule is modified to comprise one or more protective group, e.g. 5′ and/or 3′-cap structures.

As used herein, the phrase “cap structure” is meant to refer to chemical modifications that have been incorporated at either terminus of the oligonucleotide (see e.g., U.S. Pat. No. 5,998,203, incorporated by reference herein). These terminal modifications protect the nucleic acid molecule from exonuclease degradation, and can help in delivery and/or localization within a cell. The cap modification can be present at the 5′-terminus (5′-cap) or at the 3′-terminal (3′-cap), or can be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In some embodiments, the 3′-cap is selected from a group comprising inverted deoxynucleotide, such as for example inverted deoxythymidine, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (see generally Beaucage & Iyer, 1993; incorporated by reference herein).

A DNAzyme molecule can be further modified by including a 3′ cationic group, or by inverting the nucleoside at the terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

According to one embodiment, the 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the DNAzyme molecule includes a modification that improves targeting. Examples of modifications that target DNAzymes to particular bacterial cell include, but are not limited to, proteins such as cell penetrating peptides (CPP) and aptamers. Exemplary CPPs which can be used according to some embodiments of the invention are discussed in McClorey et al., Cell-Penetrating Peptides to Enhance Delivery of Oligonucleotide-Based Therapeutics, Biomedicines (2018) 6(2), 51, incorporated herein by reference. Additional CPPs are discussed herein below. Exemplary aptamers which can be used according to some embodiments of the invention are discussed in Afrasiabi et al., Therapeutic applications of nucleic acid aptamers in microbial infections, Journal of Biomedical Science (2020) volume 27, Article number: 6, incorporated herein by reference.

According to one embodiment, the DNAzyme molecule of the invention can be constructed using chemical synthesis and/or enzymatic ligation reactions using procedures known in the art (as discussed in detail below). For example, the polynucleotide can be chemically synthesized using naturally occurring nucleotides or variously modified nucleotides designed to increase the biological stability of the molecules or to increase the physical stability of the duplex formed between the polynucleotide and target nucleic acids, e.g., phosphorothioate derivatives and acridine substituted nucleotides can be used (as discussed in detail hereinabove).

The DNAzyme molecule designed according to the teachings of the present invention can be generated according to any oligonucleotide synthesis method known in the art, including both enzymatic syntheses and solid-phase syntheses. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

According to one embodiment, chemical synthesis can be achieved by the diester method, triester method, polynucleotides phosphorylase method and by solid-phase chemistry. These methods are discussed in further detail below.

Diester Method: The diester method was the first to be developed to a usable state. The basic step is the joining of two suitably protected deoxynucleotides to form a dideoxynucleotide containing a phosphodiester bond.

Triester Method: The main difference between the diester and triester methods is the presence in the latter of an extra protecting group on the phosphate atoms of the reactants and products. The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide Phosphorylase Method: This is an enzymatic method of DNA synthesis that can be used to synthesize many useful oligonucleotides. Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-Phase Methods: Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers. Equipment and reagents for executing solid-phase synthesis are commercially available from, for example, Applied Biosystems.

Phosphoramidite chemistry has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Recombinant Methods: Recombinant methods for producing nucleic acids in a cell are well known to those of skill in the art and can be implemented in cases where the DNAzyme molecule does not comprise chemical modifications. These include the use of vectors, plasmids, cosmids, and other vehicles for delivery a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Maryland; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to one embodiment, the DNAzyme molecule is formulated with or attached to a permeability enhancing moiety.

The term “permeability enhancing moiety” refers to any moiety known in the art to facilitate actively or passively or enhance permeability of the compound into the target cells (e.g. bacterial cells).

According to one embodiment, the permeability enhancing moiety is a heterologous moiety, i.e. a sequence which does not form an intrinsic part of the DNAzyme molecule. Preferably, the heterologous moiety does not affect the biological activity of the DNAzyme, i.e. silencing activity, e.g. mediating catalytic activity on the resistant gene substrate.

According to one embodiment, the permeability enhancing moiety is a proteinaceous moiety.

According to one embodiment, the permeability enhancing moiety is a non-proteinaceous moiety.

Permeability enhancing moieties include, without being limited to, lipidic moieties (i.e. naturally occurring or synthetically produced lipids) such as a cholesterol moiety (Letsinger et al, Proc. Natl. Acid. Sci. USA, 199, 86, 6553-6556), cholic acid (Manoharan et al, Biorg. Med. Chem. Let., 1994 4 1053-1060), a thioether, e.g., beryl-S-tritylthiol (Manoharan et al, Ann. N.Y. Acad. Sci., 1992, 660, 306-309; Manoharan et al, Biorg. Med. Chem. Let., 1993, 3, 2765-2770), a thiocholesterol (Oberhauser et al, Nucl. Acids Res., 1992, 20, 533-538), an aliphatic chain, e.g., dodecandiol or undecyl residues (Saison-Behmoaras et al, EMBO J, 1991, 10, 11 11-1118; Kabanov et al, FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al, Tetrahedron Lett., 1995, 36, 3651-3654; Shea et al, Nucl. Acids Res., 1990, 18, 3777-3783), a polyamine or a polyethylene glycol chain (Manoharan et al., Nucleosides and Nucleotides, 1995, 14, 969-973), or adamantane acetic acid (Manoharan et al., Tetrahedron Lett., 1995, 36, 3651-3654), a palmityl moiety (Mishra et ai, Biochim. Biophys. Acta, 1995, 1264, 229-237), an octadecylamine or hexylamino-carbonyloxycholesterol moiety (Crooke et al., J. Pharmacol. Exp. Ther., 1996, 277, 923-937), a steroid, a sphingosine, a ceramide, or a fatty acid moiety. The fatty acid moiety can be, e.g., any fatty acid which contains at least eight carbons. For example, the fatty acid can be, e.g., a nonanoyl (C.sub.9); capryl (C.sub.10); undecanoyl (C.sub.11); lauroyl (C.sub.12); tridecanoyl (C.sub.13); myristoyl (C.sub.14); pentadecanoyl (C.sub.15); palmitoyl (C.sub.16); phytanoyl (methyl substituted C.sub.16); heptadecanoyl (C.sub.17); stearoyl (C.sub.18); nonadecanoyl (C.sub.19); arachidoyl (C.sub.20); heniecosanoyl (C.sub.21); behenoyl (C.sub.22); trucisanoyl (C.sub.23); or a lignoceroyl (C.sub.24) moiety. The cell-penetrating moiety can also include multimers (e.g., a composition containing more than one unit) of octyl-glycine, 2-cyclohexylalanine, or benzolylphenylalanine. The cell-penetrating moiety contains an unsubstituted or a halogen-substituted (e.g., chloro) biphenyl moiety. Substituted biphenyls are associated with reduced accumulation in body tissues, as compared to compounds with a non-substituted biphenyl. Reduced accumulation in bodily tissues following administration to a subject is associated with decreased adverse side effects in the subject.

According to one embodiment, the permeability enhancing moiety is a peptide (e.g. cell penetrating peptide (CPP)). Suitable peptides typically include short (typically less than 30 amino acids) amphipathic or cationic peptide fragments that are typically derived from naturally occurring protein translocation motifs, as in the case of HIV-TAT (transactivator of transcription protein), Penetratin 1 (homeodomain of the Drosophila Antennapedia protein) and Transportan (a chimeric peptide consisting of part of the galanin neuropeptide fused to the wasp venom, mastoparan), or are based on polymers of basic amino acids (e.g. arginine and lysine). Additional examples are provided in Roberts et al. Nature Reviews Drug Discovery (2020), incorporated herein by reference.

According to one embodiment, the permeability enhancing moiety is a polysaccharide (e.g. mannose), a synthetic nucleoside base, an inverted nucleoside base, a cholesterol, other sterols (e.g. methyl sterols, dimethyl sterols), a lipid, a membrane lipid (e.g. phospholipids, glycolipids), and a synthetic lipid.

The DNAzyme molecule and the permeability enhancing moiety may be coupled directly or indirectly via an intervening moiety or moieties, such as a linker, a bridge, or a spacer moiety or moieties.

According to one embodiment, the DNAzyme molecule and the permeability enhancing moiety may be directly coupled. Alternatively, according to another embodiment, the moiety may be linked by a connecting group. The terms “connecting group”, “linker”, “linking group” and grammatical equivalents thereof are used herein to refer to an organic moiety that connects two parts of a compound.

The permeability enhancing moiety can be attached to any nucleotide in the DNAzyme molecule, but it can be preferably coupled through the 3′ terminal nucleotide and/or 5′ terminal nucleotide. An internal conjugate may be attached directly or indirectly through a linker to a nucleotide at a 2′ position of the ribose group, or to another suitable position.

Thus, the permeability enhancing moiety can be attached to any nucleotide within the DNAzyme molecule as long as the silencing activity (e.g. mediating catalytic activity) of the DNAzyme is not compromised.

Any of the above described moieties (e.g. permeability enhancing moiety) may be selected by the skilled person taking into consideration the target tissue, the target cell, the administration route, the pathway that the DNAzyme is expected to follow, etc.

According to one embodiment, when the moiety (e.g. permeability enhancing moiety) is a peptide or peptide product, it may be subjected to in-vitro modification (e.g., PEGylation, lipid modification, etc.) so as to confer the peptide's amino acid sequence with stability (e.g., against protease activities) and/or solubility (e.g., within a biological fluid such as blood, digestive fluid) while preserving its biological activity and prolonging its half-life.

When the DNAzyme molecule is coupled to a permeability enhancing moiety, synthesis can be carried out using standard procedures in organic synthesis. The skilled person will appreciate that the exact steps of the synthesis will depend on the exact structure of the molecule which has to be synthesized. For instance, if the molecule is attached to the permeability enhancing moiety through its 5′ end, then the synthesis is usually carried out by contacting an amino-activated oligonucleotide and a reactive activated permeability enhancing moiety.

According to one embodiment, the DNAzyme is coupled to a permeability enhancing moiety and to a protecting group (e.g. the permeability enhancing moiety is coupled to the 5′ end of the DNAzyme and the protecting group to the 3′ end, or vice versa).

According to a specific embodiment, the nucleic acid oligonucleotide is conjugated to a permeability enhancing moiety is cholesterol. According to one embodiment, the cholesterol is linked directly or via a linker to the 5′ or 3′ terminus (or both) of the oligonucleotide.

The DNAzyme molecules of the invention are to be provided to the cells i.e., target cells (e.g. bacterial cells) of the present invention in vivo (i.e., inside a subject having a bacterial infection), in vitro or ex vivo (e.g., in a tissue culture or flask). It will be appreciated that the DNAzyme molecule may be provided directly to the target cells, or alternatively may be administered to a tissue or organ comprising the target cells, or to a subject in need of eradication of bacterial cells.

For example, for in vitro or ex vivo applications, any laboratory testing applications can be utilized to assess the efficacy of the DNAzyme molecules of the invention on rending the bacteria susceptible to antibiotic treatment. An exemplary method includes the disk diffusion test used to determine the susceptibility of clinical isolates of bacteria to different antibiotics. Accordingly, the bacteria may be contacted with the DNAzyme molecule and the antibiotics concomitantly or subsequent to each other (e.g. within minutes, hours or days). Such determinations are well within the skill of a person skilled in the art.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009: 410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

According to one embodiment, the present techniques relate to introducing the DNAzyme molecules using transient DNA or DNA-free methods (such as transfection).

According to one embodiment, the DNAzyme molecule is delivered as a “naked” oligonucleotide, i.e. without the additional delivery vehicle. According to one embodiment, the “naked” oligonucleotide comprises a chemical modification to facilitate its tissue delivery (e.g. utilizing inverted nucleotides, phosphorothioate linkages, or integration of locked nucleic acids, as discussed above).

Any method known in the art for RNA or DNA transfection can be used in accordance with the present teachings, such as, but not limited to microinjection, electroporation, lipid-mediated transfection e.g. using liposomes, or using cationic molecules or nanomaterials (discussed below, and further discussed in Roberts et al. Nature Reviews Drug Discovery (2020) 19: 673-694, incorporated herein by reference).

According to one embodiment, in cases where the DNAzyme molecule does not comprise a chemical modification it may be administered to the target cell (e.g. bacterial cell) as part of an expression construct. In this case, the DNAzyme molecule is ligated in a nucleic acid construct (e.g. as part of a “vector” or an “expression vector” as discussed below) under the control of a cis-acting regulatory element (e.g. promoter) capable of directing an expression of the DNAzyme in the target cells (e.g. bacterial cell) in a constitutive or inducible manner.

The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, sequence which is able to transform host cells and optionally capable of replicating in a host cell. The vector may contain an optional marker suitable for use in the identification of transformed cells, e.g., tetracycline resistance or ampicillin resistance. A cloning vector may or may not possess the features necessary for it to operate as an expression vector.

According to one embodiment, the vector is a plasmid. According to another embodiment, the vector is a phage.

According to one embodiment, DNAzyme oligonucleotides are obtained upon replication or transcription of the vector (e.g. in the bacterial cell).

According to one embodiment, the vector comprises a nucleic acid construct comprising the sequences of the DNAzyme oligonucleotides of some embodiments of the invention.

According to one embodiment, the DNAzyme oligonucleotides is operably linked to an origin of replication and to a termination site. If multiple DNAzyme oligonucleotides are utilized, each one of the sequences of the DNAzyme oligonucleotides is operably linked to an origin of replication and to a termination site.

The term “operably linked” as used herein refers to an arrangement of elements that allows them to be functionally related. Thus, in one embodiment, the one or more DNAzyme oligonucleotides is operably linked to an origin of replication and to a termination site such that the DNAzyme oligonucleotides is separately replicated by the bacterial DNA replication machinery.

According to another embodiment, the vector comprises a nucleic acid construct comprising the sequences of the DNAzyme oligonucleotides of some embodiments of the invention, wherein the nucleic acid construct is operably linked to an origin of replication and to a termination site, and wherein the nucleic acid construct comprises a cleavable sequence between every pair of oligonucleotides' sequences. According to one embodiment, the cleavable sequence is a hairpin-forming sequence. According to some embodiments, the hairpin is an enzymatically cleavable hairpin. According to such embodiments, the nucleic acid comprising the sequences of the DNAzyme oligonucleotides is replicated by the bacterial DNA replication machinery and consequently spliced or parsed either via self-splicing or via enzymatic splicing to produce DNA DNAzyme oligonucleotides.

According to one embodiment, the vector comprises a nucleic acid construct comprising the reverse sequences of the DNAzyme oligonucleotides of some embodiments of the invention. According to one embodiment, the each one of the reverse sequences of the DNAzyme oligonucleotides is operably linked to a promoter. According to such embodiments, each reverse sequences of DNAzyme oligonucleotides is separately transcribed by the bacterial transcription machinery to produce RNA DNAzyme oligonucleotide.

According to another embodiment, the vector comprises a nucleic acid construct comprising the reverse sequences of the DNAzyme oligonucleotides of some embodiments of the invention, wherein the nucleic acid construct is operably linked to a promoter, and wherein the nucleic acid construct encodes for a cleavable sequence between every pair of oligonucleotides' sequences. According to one embodiment, the cleavable sequence is a hairpin-forming sequence. According to some embodiments, the hairpin is an enzymatically cleavable hairpin. According to such embodiments, the nucleic acid comprising the sequences of the DNAzyme oligonucleotides is transcribed by the bacterial transcription machinery and consequently spliced or parsed either via self-splicing or via enzymatic splicing to obtain RNA DNAzyme oligonucleotides.

The expression constructs of the present invention may also include additional sequences which render it suitable for replication and integration in eukaryotes (e.g., shuttle vectors). Typical cloning vectors contain transcription and translation initiation sequences (e.g., promoters, enhances) and transcription and translation terminators (e.g., polyadenylation signals). The expression constructs of the present invention can further include an enhancer, which can be adjacent or distant to the promoter sequence and can function in up regulating the transcription therefrom. Polyadenylation sequences can also be added to the expression constructs of the present invention in order to increase the efficiency of expression.

In addition to the embodiments already described, the expression constructs of the present invention may typically contain other specialized elements intended to increase the level of expression of cloned nucleic acids or to facilitate the identification of cells that carry the DNAzyme. The expression constructs of the present invention may or may not include a eukaryotic replicon.

According to a further aspect, the present invention provides a vector comprising the nucleic acid construct of the present invention.

The expression construct may be introduced into the target cells (e.g. bacterial cells) of the present invention using an appropriate gene delivery vehicle/method (transfection, transduction, etc.) and an appropriate expression system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, the oligonucleotide is encapsulated in a liposome, encapsulated in a viral capsid, conjugated to a micro- or nano-particle, or embedded in a polymer matrix such as gel, PLGA, PEG, etc.

Suitable viral capsids include, but are not limited to, AAV (Adeno-associated virus) capsid, MS2 bacteriophage capsid, HBV (hepatitis B virus) capsid, and CCMV (Cowpea chlorotic mottle virus) capsid.

Lipid-based systems which may be used for the delivery of constructs or DNAzyme molecules into the target cells (e.g. bacterial cells) of the present invention. Lipid bases systems include, for example, liposomes, lipoplexes and lipid nanoparticles (LNPs).

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43]. Any method known in the art can be used to incorporate a polynucleotide agent (e.g. DNAzyme molecule) into a liposome. For example, the polynucleotide agent (e.g. DNAzyme molecule) may be encapsulated within the liposome. Alternatively, it may be adsorbed on the liposome's surface. Other methods that may be used to incorporate a pharmaceutical agent into a liposome of the present invention are those described by Alfonso et al., [The science and practice of pharmacy, Mack Publishing, Easton Pa 19^{th} ed., (1995)] and those described by Kulkarni et al., [J. Microencapsul. 1995, 12 (3) 229-46].

Furthermore, lipid nanoparticles (LNPs), also known as stable nucleic acid lipid particles, may be used in accordance with the present teachings. These are typically liposomes that contain ionizable lipid, phosphatidylcholine, cholesterol and PEG-lipid conjugates, as discussed in Roberts et al. Nature Reviews Drug Discovery (2020), incorporated herein by reference.

The lipid bases systems (e.g. liposomes) used in the methods of the present invention may cross the blood barriers. Thus, according to an embodiment the lipid bases systems (e.g. liposomes) of the present invention do not comprise a blood barrier targeting polysaccharide (e.g. mannose) in their membrane portion. In order to determine lipid bases systems (e.g. liposomes) that are especially suitable in accordance with the present invention a screening assay can be performed such as the assays described in U.S. Pat. Appl. No. 20040266734 and U.S. Pat. Appl. No. 20040266734; and in Danenberg et al., Journal of cardiovascular pharmacology 2003, 42:671-9; Circulation 2002, 106:599-605; Circulation 2003, 108:2798-804.

For in vivo therapy, the composition of matter of some embodiments of the invention comprising a DNAzyme molecule is administered to the subject per se or as part of a pharmaceutical composition.

According to another aspect the present invention there is provided a pharmaceutical composition comprising at least one of (i) at least one DNAzyme of some embodiments the present invention; (ii) at least one nucleic acid construct comprising the sequences or reverse sequences of the at least one DNAzyme oligonucleotides; or (iii) at least one vector of the present invention; and a pharmaceutically acceptable excipient.

In some embodiments of the pharmaceutical composition of matter, the composition is comprised of 1 (one) oligonucleotide DNAzyme, 2 (two) oligonucleotide DNAzymes, or more in varying ratios.

In some embodiments of the pharmaceutical composition of matter, the composition is comprised of at least one oligonucleotide DNAzyme and at least one antibiotic compound.

According to a further embodiment, the present invention provides a pharmaceutical composition comprising the at least one vector comprising the nucleic acid DNAzyme, such that the vector is introduced into the bacterial cell, in which it encodes the active compound as a DNA or RNA oligonucleotide.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the DNAzyme molecule accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include systemic, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to a specific embodiment, the composition is for inhalation mode of administration.

According to a specific embodiment, the composition is for intranasal mode of administration.

According to a specific embodiment, the composition is for intracerebroventricular mode of administration.

According to a specific embodiment, the composition is for intrathecal mode of administration.

According to a specific embodiment, the composition is for oral mode of administration.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to a specific embodiment, the composition is for local injection.

According to a specific embodiment, the composition is for systemic administration.

According to a specific embodiment, the composition is for intravenous administration.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the present invention include compositions wherein the active ingredients are contained in an amount effective to achieve the intended purpose. More specifically, a therapeutically effective amount means an amount of active ingredients (e.g. DNAzyme molecule) effective to prevent, alleviate or ameliorate symptoms of a disorder (e.g., bacterial infection) or prolong the survival of the subject being treated.

According to an embodiment of the present invention, administration of the DNAzyme molecule has a bactericidal effect (i.e. increases susceptibility to antibiotic treatment).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide sufficient levels of the active ingredient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that animal models exist by which the DNAzyme molecules of the present invention may be tested prior to human treatment. For example, animal models for testing DNAzyme toxicity and effectivity include larvae and thigh muscle infection model in neutropenic mice (as discussed in detail in the general materials and experimental procedures section, below).

Furthermore, formulation of the pharmaceutical composition may be adjusted according to applications. In particular, the pharmaceutical composition may be formulated using a method known in the art so as to provide rapid, continuous or delayed release of the active ingredient after administration to mammals.

According to any one of the above embodiments, the pharmaceutical composition is formulated to enhance the permeability of the oligonucleotide into bacterial cells.

In some embodiments of the pharmaceutical composition, the composition includes a combination of 1, 2, or more DNAzyme oligonucleotides.

In some embodiments of the pharmaceutical composition, the composition includes a combination of 1, 2, or more DNAzyme oligonucleotides, and an antibiotic compound such as penicillin, methicillin, oxacillin, cefoxitin, cephalosporin, carbapenem, imipenem, meropenem, aztreonam, etc. (discussed in further detail herein above and below).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, there is provided an article of manufacture comprising the composition of matter of some embodiments of the invention, being packaged in a packaging material and identified in print, in or on the packaging material for use in the treatment of bacterial infection.

It will be appreciated that the therapeutic compositions of the invention may comprise, in addition to the DNAzyme molecule, other known medications for the treatment of bacterial infections, e.g. antibacterial agents such as antibiotics. Exemplary antibiotics which can be used in accordance with some embodiments of the invention include, but are not limited to, penicillins (e.g., oxacillin, methicillin, amoxicillin and amoxicillin-clavulanate), monobactams (e.g aztreonam), clavulanate acid, trimethoprim-sulfamethoxazole, cephalosporins (e.g., third-generation oxyimino-cephalosporins and methoxy-cephalosporins, including but not limited to, ceftazidime, cefotaxime, cefuroxime, ceflacor, cefprozil, loracarbef, cefindir, cefixime, cefpodoxime proxetil, cefibuten, ceftriaxone, cephamycins (e.g. cefoxitin), carbapenems (e.g. imipenem-cilastatin, meropenem, ertapenem, doripenem, panipenem-betamipron, and biapenem)), fluoroquinolone (e.g., ofloxacin, ciprofloxacin, levofloxacin, trovafloxacin), macrolides, azalides (e.g., erythromycin, clarithromycin, and azithromycin), sulfonamides, ampicillin, tetracycline, chloramphenicol, minocycline, doxycycline, vancomycin, bacitracin, kanamycin, neomycin, gentamycin, erythromycin, spectinomycin, zeomycin, streptomycin as well as any of combinations and any derivatives thereof.

According to a specific embodiment, the antibiotic is a β-lactam.

According to a specific embodiment, the antibiotic is a Carbapenem.

According to a specific embodiment, the antibiotic is a Penicillin.

According to a specific embodiment, the antibiotic is a Cephalosporin.

According to a specific embodiment, the antibiotic is a Monobactam.

Any of the above described compositions may be packed together or separately (e.g. in a single container or in separate containers): e.g., DNAzyme packed separately from the antibiotic; or DNAzyme and the antibiotic in a single container.

As mentioned above, the DNAzyme of some embodiments of the invention is capable of increasing susceptibility of bacteria to antibiotic therapy. Accordingly, the DNAzymes may be used alone, or with antibiotics, to treat bacterial infections.

According to another aspect, the present invention provides a method of treating a bacterial infection in a subject in a need thereof comprising administering to the subject the pharmaceutical composition of some embodiments of the present invention. According to some embodiments, the pharmaceutical composition comprising at least one of (i) at least one DNAzyme oligonucleotides of the present invention; (ii) at least one nucleic acid construct comprising the sequences or reverse sequence of some embodiments of the invention; or (iii) at least one vector of the present invention; and a pharmaceutically acceptable excipient.

According to another aspect of the present invention, there is provided a method of treating or preventing a bacterial infection in a subject in need thereof, the method comprising administering to the subject one or more DNAzyme oligonucleotides of some embodiments of the invention, or one or more of the nucleic acid constructs of some embodiments of the invention.

According to another aspect of the present invention, there is provided an oligonucleotide of some embodiments of the invention, or nucleic acid construct of some embodiments of the invention, for use in treating or preventing a bacterial infection in a subject in need thereof.

According to another aspect of the present invention, there is provided a use of an oligonucleotide of some embodiments of the invention, or nucleic acid construct of some embodiments of the invention, for treating or preventing a bacterial infection in a subject in need thereof.

According to another aspect of the present invention, there is provided a method of treating or preventing a bacterial infection in a subject in need thereof, the method comprising administering to the subject one or more DNAzyme oligonucleotides of some embodiments of the invention, or one or more of the nucleic acid constructs of some embodiments of the invention, and an antibiotic.

According to another aspect of the present invention, there is provided an oligonucleotide of some embodiments of the invention, or nucleic acid construct of some embodiments of the invention, and an antibiotic for use in treating or preventing a bacterial infection in a subject in need thereof.

According to another aspect of the present invention, there is provided a use of an oligonucleotide of some embodiments of the invention, or nucleic acid construct of some embodiments of the invention, and an antibiotic for treating or preventing a bacterial infection in a subject in need thereof.

The term “treating” refers to inhibiting, preventing or arresting the development of a pathology (disease, disorder or condition) and/or causing the reduction, remission, or regression of the pathology. Those of skill in the art will understand that various methodologies and assays can be used to assess the development of a pathology, and similarly, various methodologies and assays may be used to assess the reduction, remission or regression of a pathology.

According to one embodiment, treating results in killing bacteria as well as preventing bacteria growth and replication.

As used herein, the term “subject” or “subject in need thereof” includes mammals, such as human beings, male or female, at any age which suffers from the pathology or is at risk to develop the pathology.

According to a specific embodiment, the subject is a human subject.

According to a specific embodiment, the subject is a non-human subject (e.g. a farm animal, e.g. a mammal including e.g. a horse, a donkey, a pig, a sheep, a goat, a cow, a dog, a cat, a rabbit, a rat, a hamster, a mouse; a chicken, a duck, a goose).

The term “bacterial infection” relates to any disease or disorder caused directly or indirectly by bacteria. Bacterial infection typically causes the symptoms of fever, chills, exhaustion, headache, swollen lymph nodes, skin flushing/swelling/soreness and/or gastrointestinal symptoms, such as nausea, vomiting, diarrhea, abdominal or rectal pain. Such symptoms may persist for several hours, days or weeks.

According to one embodiment, the bacterial infection is caused by gram-negative bacteria (discussed in detail above).

According to one embodiment, the bacterial infection is caused by gram-positive bacteria (discussed in detail above).

According to one embodiment, the bacterial infection is caused by a single microbial species.

According to one embodiment, the bacterial infection is caused by a mixed species complexes (e.g. by two or more bacterial species).

According to one embodiment, the bacterial infection is caused by antibiotic-resistant bacteria.

According to a specific embodiment, the bacterial infection is caused by an Enterococcus faecium, a Staphylococcus aureus, a Klebsiella pneumoniae, an Acinetobacter baumannii, a Pseudomonas aeruginosa or an Enterobacter.

According to a specific embodiment, the bacterial infection is caused by Klebsiella pneumoniae.

According to a specific embodiment, the bacterial infection is caused by Staphylococcus aureus.

According to a specific embodiment, the bacterial infection is caused by methicillin-resistant Staphylococcus aureus (MRSA).

According to a specific embodiment, the bacteria is Pseudomonas aeruginosa.

Non-limiting examples of diseases and disorders caused by bacteria that may be treated by the methods of some embodiments of the invention include, but are not limited to, actinomycosis, anaplasmosis, anthrax, bacillary angiomatosis, actinomycetoma, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cat-scratch disease, cellulitis, chancroid, chlamydia, chlamydia conjunctivitis, clostridial myonecrosis, cholera, Clostridium difficile colitis, diphtheria, Daintree ulcer, donavanosis, dysentery, erhlichiosis, epidemic typhus, fried rice syndrome, five-day fever, floppy baby syndrome, Far East scarlet-like fever, gas gangrene, glanders, gonorrhea, granuloma inguinale, human necrobacillosis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, plague, pneumococcal infection, pneumonia, psittacosis, parrot fever, pseudotuberculosis, Q fever, quintan fever, rabbit fever, relapsing fever, rickettsialpox, Rocky Mountain spotted fever, rat-bite fever, Reiter syndrome, rheumatic fever, salmonellosis, scarlet fever, sepsis, septicemic plague, Searls ulcer, shigellosis, soft chancre, syphilis, streptobacillary fever, scrub typhus, Taiwan acute respiratory agent, Trench fever, trachoma, tuberculosis, tularemia, typhoid fever, typhus, tetanus, toxic shock syndrome, undulant fever, Ulcus molle, Vibrio parahaemolyticus enteritis, Whitmore's disease, walking pneumonia, Waterhouse-Friderichsen syndrome, yaws, and yersiniosis.

According to one embodiment, treating the bacterial infection pertains to treating the inflammatory disorder caused by the bacterial infection. Non-limiting examples of such inflammatory disorders include, but are not limited to, adenoiditis, appendicitis, arteritis, ascending cholangitis, balanitis, blepharitis, bronchitis, bursitis, cellulitis, cerebral vasculitis, cervicitis, chemosis, cholecystitis, chondritis, choroioamnionitis, colitis, conjunctivitis, constrictive pericarditis, cryptitis, dacryoadenitis, dermatitis, duodenal lymphocytosis, encephalitis, endocarditis, endometritis, endotheliitis, enteritis, enterocolitis, eosinophilis fasciitis, epididymitis, esophagitis, folliculitis, gastritis, gingivitis, glomerulonephritis, glossitis, hepatitis, infectious arthritis, ileitis, intertrigo, keratitis, keratoconjunctivitis, labyrithitis, lymphadenitis, mastitis, mastoiditis, myocarditis, myopericarditis, myositis, necrotizing fasciitis, nephritis, omaphalitis, oophoritis, ophthalmitis, orchitis, osteitis, osteomyelitis, pancreatitis, paraproctitis, parotitis, pericarditis, perichondritis, perifolliculitis, periodontitis, peritonitis, pharyngitis, phlebitis, pleurisy, pneumonitis, pulmonitis, proctitis, prostatitis, pulpitis, pyelonephritis, pyomyositis, retinal vasculitis, rheumatic fever, rhinitis, scleritis, salpingitis, sialadenitis, sinusitis, stomatitis, synovitis, septicemia, tenosynovitis, thyroiditis, tonsillitis, tularemia, urethritis, uveitis, vaginitis, vasculitis, and vulvitus.

According to a specific embodiment, the bacterial infection is a pneumonia, a urinary tract infection and a sepsis.

As mentioned, the DNAzyme oligonucleotide, nucleic acid construct or vector of some embodiments of the invention is typically utilized with an antibiotic. A non-limiting list of antibiotics is provided herein above. Additional suitable antibiotics for use in the context of some embodiments of the present invention include, but are not limited to, amikacin, amoxicillin, ampicillin, azithromycin, aztreonam, carbapenem, cefazolin, ceftriaxone, cefepime, cefonicid, cefotetan, cefoxitin, ceftazidime, cephalosporin, cephamycin, chloramphenicol, chlortetracycline, ciprofloxacin, clarithromycin, clindamycin, colistin, cycloserine, dalfopristin, doxycycline, ephalothin, erythromycin, gatifloxacin, gentamicin, imipenem, kanamycin, levofloxacin, lincosamide, linezolid, meropenem, methicillin, moxifloxacin, mupirocin, neomycin, nitrofurantoin, oxacillin, oxytetracycline, piperacillin, penicillin, quinupristin, rifampicin, spectinomycin, streptomycin, sulfanilamide, sulfamethoxazole, tazobactam, tetracycline, tobramycin, trimethoprim and vancomycin, as well as any of combinations and any derivatives thereof. The antibiotic and the DNAzyme oligonucleotide, nucleic acid construct or vector may be administered to the subject (e.g. to a human subject) concomitantly, concurrently, simultaneously, consecutively or sequentially with one another.

According to one embodiment, the antibiotic and the DNAzyme oligonucleotide, nucleic acid construct or vector are in separate formulations.

According to one embodiment, the antibiotic and the DNAzyme oligonucleotide, nucleic acid construct or vector are in a co-formulation.

According to one embodiment, when more than one DNAzyme is utilized (e.g. 2, 3, or more), these may be administered to the subject (e.g. to a human subject) concomitantly, concurrently, simultaneously, consecutively or sequentially with one another. Similarly, when more than one antibiotic compound is utilized (e.g. 2, 3, or more), these may be administered to the subject (e.g. to a human subject) concomitantly, concurrently, simultaneously, consecutively or sequentially with one another. Such determinations are well within the skill of a person skilled in the art.

Those of skill in the art will understand that various methodologies and assays can be used to assess the efficiency of treatment, for example, by doctor examination, by assessing temperature and blood pressure of the subject, and by detecting the levels of bacterial cells or products (e.g. toxins) in biological samples (e.g. blood, serum, urine, skin sample, cerebrospinal fluid (CSF)) by using, for example, a bacteria culture test, blood or urine test.

According to one embodiment, an efficient anti-bacterial treatment is determined when there is a decrease of at least about 10%, 20%, 30%, 40%, 50%, 60%, 70%, 80%, 90% or 100% or more in the number of bacterial cells or bacterial products (e.g. toxins), as compared to the number of bacterial cells or products in the subject being treated but prior to the treatment.

Furthermore, an efficient anti-bacterial treatment is determined when there is a decrease in fever back to normal levels, and pain levels decrease and general wellbeing is improved. Such determinations are well known to persons of skill in the art.

According to another embodiment, there is provided a surface coated with the oligonucleotide of some embodiments of the invention, or nucleic acid construct or vector of some embodiments of the invention.

According to another embodiment, there is provided a surface coated with the oligonucleotide of some embodiments of the invention, or nucleic acid construct or vector of some embodiments of the invention, and an antibiotic.

The term “surface” is defined herein as any surface which may be covered, at least in part, by bacteria (e.g. by biofilm). Exemplary surfaces include, but not limited to, fabrics, fibers, foams, films, concretes, masonries, glass, metals, plastics, rubbers, polymers, combinations of same and like.

The present invention contemplates coating of organic surfaces, inorganic surfaces, or combinations of same.

The present invention contemplates coating of medical devices susceptible to biofilm formation.

According to one embodiment, the surface does not comprise bacteria (and said treatment is effected as a preventive measure).

According to another embodiment, the surface already has bacteria attached thereto.

Coating a surface can be effected using any method known in the art including, but not limited to, spraying, spreading, wetting, immersing, dipping, painting, ultrasonic welding, welding, bonding or adhering.

Coating may be affected as monolayers or multiple layers.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Oligonucleotides

DNA oligonucleotides, including any modification, were ordered from Integrated DNA Technologies (TDT), reconstituted to 100 μM with ultrapure, DNase/RNase free water (Biological Industries, Israel), and stored at −20° C. DNA sequences are listed in Tables 1 and 2, below.

TABLE 1
DNAzymes effective against Klebsiella

| | | SEQ ID |
|---|---|---|
| DNAzyme | Sequence | NO |
| KPC_337 | GCCTGTTGggctagctacaacgaCAGATATTT/3CholTEG/ | 1 |
| KPC_332 | GTTGTCAGAggctagctacaacgaATTTTTCCG/3CholTEG/ | 2 |
| SHV-1_133 | CCAGATCCAggctagctacaacgaTTCTATCAT/3CholTEG/ | 3 |
| TEM_588 | TAGAGTAAGggctagctacaacgaAGTTCGCC/3CholTEG/ | 4 |
| KPC_568 | CAGTTTTTGggctagctacaacgaAAGCTTTCC/3CholTEG/ | 35 |
| KPC_36 | CATGAGAGAggctagctacaacgaAAGACAGCA/3CholTEG/ | 36 |
| KPC_470 | GAACGTGGggctagctacaacgaATCGCCGA/3CholTEG/ | 37 |
| KPC_389 | CGGCGTTAggctagctacaacgaCACTGTATT/3CholTEG/ | 38 |
| KPC_563 | TTTTGTAAGggctagctacaacgaTTTCCGTCA/3CholTEG/ | 39 |
| KPC_332 | GTTGTCAGAggctagctacaacgaATTTTTCCG/3CholTEG/ | 40 |
| KPC_574 | CAGTGTCAGggctagctacaacgaTTTTGTAAG/3CholTEG/ | 41 |
| KPC_633 | GTGTTTCCtccgagccggacgaTTAGCCAAT/3CholTEG/ | 42 |
| KPC_344 | CCGTCATGggctagctacaacgaCTGTTGTCA/3CholTEG/ | 43 |
| OXA-18_294 | ATAGACTTGggctagctacaacgaTTGTATGTG/3CholTEG/ | 44 |
| OXA-18_59 | CCACGGAAggctagctacaacgaTGATTGGGA/3CholTEG/ | 45 |
| OXA-18_125 | TATAAGGTAggctagctacaacgaTTCCGGTAA/3CholTEG/ | 46 |
| OXA-18_225 | TTTGGCGAggctagctacaacgaTGCAAGATT/3CholTEG/ | 47 |
| SHV-1_127 | CCATTTCTAggctagctacaacgaCATGCCTAC/3CholTEG/ | 48 |
| SHV-1_33 | GGTGGCTAAggctagctacaacgaAGGGAGATA/3CholTEG/ | 49 |
| SHV-1_197 | TTAAAGGTGggctagctacaacgaTCATCATGG/3CholTEG/ | 50 |
| TEM_518 | GCTCGTCGggctagctacaacgaTTGGTATGG/3CholTEG/ | 51 |
| TEM_810 | TCTATTTCGggctagctacaacgaTCATCCATA/3CholTEG/ | 52 |
| TEM_14 | ACACGGAAAggctagctacaacgaGTTGAATAC/3CholTEG/ | 53 |

TABLE 2
DNAzymes effective against MRSA

| | | SEQ ID |
|---|---|---|
| DNAzyme | Sequence | NO |
| USA300HOU_ | TTTTAGTTGggctagctacaacgaGTTAGTACT/3CholTEG/ | 5 |
| 2333_1302_9nt | | |
| mecA_650_10nt | TATTTTAGCAggctagctacaacgaAGTCATTTAA/3CholTEG/ | 6 |
| mecA_661_10nt | TGACTCATAAggctagctacaacgaTATTTTAGCA/3CholTEG/ | 7 |
| mecA-658_11nt | TGACTCATAATggctagctacaacgaTTTAGCATAGT/3CholTEG/ | 8 |
| USA300HOU_ | GCTTTTTTAggctagctacaacgaTGACTAATG/3CholTEG/ | 9 |
| 2396: 437_9nt | | |
| USA300HOU_ | AGCTTTTTTAggctagctacaacgaTGACTAATGG/3CholTEG/ | 10 |
| 2396: 437_10nt | | |
| mecA_647_9nt | TTAGCATAGggctagctacaacgaCATTTAAAT/3CholTEG/ | 11 |
| mecA_647-11nt | TTTTAGCATAGggctagctacaacgaCATTTAAATAA/3CholTEG/ | 12 |
| mecA_650_9nt | ATTTTAGCAggctagctacaacgaAGTCATTTA/3CholTEG/ | 13 |
| mecA_650_11nt | TTATTTTAGCAggctagctacaacgaAGTCATTTAAA/3CholTEG/ | 14 |
| mecA_661_9nt | GACTCATAAggctagctacaacgaTATTTTAGC/3CholTEG/ | 15 |
| glpT_1122_9nt | TTTAGTAAGggctagctacaacgaTCTTTATGG/3CholTEG/ | 16 |
| glpT_1122_10nt | ATTTAGTAAGggctagctacaacgaTTATGGGTAC/3CholTEG/ | 17 |
| USA300HOU_ | TTTTAGTTGggctagctacaacgaGTTAGTACT/3CholTEG/ | 18 |
| 2333_1302_9nt | | |
| USA300HOU_ | TTTTTAGTTGggctagctacaacgaGTTAGTACTC/3CholTEG/ | 19 |
| 2333_1302_10nt | | |
| mecA-658_9nt | TCATAATTAggctagctacaacgaTTTAGCATA/3CholTEG/ | 20 |
| mecR1_146 | GAAGTCGTGggctagctacaacgaCAGATACAT/3CholTEG/ | 21 |
| mecA_353 | GTTCGTTGtccgagccggacgaCGAATAATT/3CholTEG/ | 22 |
| USA300HOU_ | GCTTTTTTAggctagctacaacgaTGACTAATG/3CholTEG/ | 23 |
| 2396: 437 | | |
| USA300HOU_ | ATTTATTTAggctagctacaacgaAAAATTTAC/3CholTEG/ | 24 |
| 2333: 676 | | |
| femA_545 | TTTTTCGTGggctagctacaacgaTTCTTTTTC/3CholTEG/ | 25 |
| mecA_658_9nt | TCATAATTAggctagctacaacgaTTTAGCATA/3CholTEG/ | 26 |

Bacterial Strains

Klebsiella pneumoniae (KP, ATCC® BAA-1705™), Staphylococcus aureus (ATCC® BAA-1717™) and Pseudomonas aeruginosa (PA, ATCC® BAA-3105™) were purchased from ATCC, streaked onto Luria broth (LB) agar plates (HyLabs, Israel) and grown for 24 hours at 37° C. A single colony from each strain was arbitrarily selected and frozen in LB supplemented with 30% glycerol (HyLabs, Israel) and stored at −80° C. for all assays.

Flow Cytometry

Flow cytometry was performed on a Becton-Dickinson Accuri C6 Plus cytometer equipped with 488 nm solid-state laser and a 640 nm diode laser. Data was analyzed using Kaluza Analysis 2.1 software using a C6 import module. For measurements of intracellular fluorescent DNAzyme, bacterial strains were streaked from frozen LB+glycerol stocks onto LB agar plates and incubate for 24 hours at 37° C. Single colonies were grown overnight at 37° C. in 3 ml of LB. Cultures were diluted 1:100 in 2 ml LB and grown till mid log (OD 0.6-0.8) at 37° C. Fluorescent DNAzyme were added to a final concentration of 1.25 μM. Cells were harvested every 30 minutes, washed twice with 1× Dulbecco's Phosphate Buffered Saline (PBS, Biological Industries) and were treated with DNase I (New England Biolabs) for 5 minutes in 1×PBS at room temperature and washed again with 1×PBS. Data were collected from approximately 50,000 cells per time point using the FL-4 laser.

Growth Assays

Bacterial strains were streaked from frozen LB+glycerol stocks onto LB agar plates and incubate for 24 hours at 37° C. Single colonies were grown overnight at 37° C. in 3 ml of LB. Cultures were diluted 1:1000 in 2 ml LB supplemented with 128 μg/ml meropenem and DNAzyme were added to a final concentration of 1.25 μM. Cultures were incubated at 37° C. with continuous shaking and the optical density OD₆₀₀ was recorded every 30 minutes using Ultrospec 10 (biochrom). Viability was tested by serial dilutions (1:100) of the bacterial suspensions and spots were plated on LB plates. The plates were incubated at 37° C. and on the next day CFU was counted to determine culture viability.

Bla-KPC Quantification from Bacterial Cells

Bacterial strains were streaked from frozen LB+glycerol stocks onto LB agar plates and incubate for 24 hours at 37° C. Single colonies were grown overnight at 37° C. in 3 ml of LB. Cultures were diluted 1:1000 in 2 ml LB supplemented with 128 μg/ml meropenem and DNAzyme were added to a final concentration of 1.25 μM. Cultures were incubated at 37° C. with continuous shaking for 20 minutes, then 1 ml of the culture were added to 1 ml of RNA protect (Qiagen). Total RNA was extracted from each sample using RNeasy Mini Kit (Qiagen), according to the manufacturer's instructions. The concentration, purity, and integrity of total RNA was determined using NanoDrop and gel electrophoresis. cDNA synthesis was performed using iScript™ cDNA synthesis (Bio-Rad) according to the manufacturer's instructions. Equal amounts of total RNA (1000 ng/20 μL) were reverse-transcribed in all samples. Reactions were incubated in a CFX96 Touch Real-Time PCR detection system (Bio-Rad) by the following program: 25° C. for 5 minutes, 46° C. for 20 minutes, 95° C. for 60 seconds. Bla-KPC expression was determined by qPCR, with primers designed using NCBI primer-BLAST. qPCR analysis was performed using the iTaq Universal SYBR Green Supermix (Bio-Rad) according to the manufacturer's instructions, in a CFX96 system by the following program: 95° C. for 3 minutes, 39 cycles of 95° C. for 10 seconds and 55° C. for 30 seconds. Melt curves were generated for each sample by heating PCR amplicons from 65° C. to 95° C. with a gradual increase of 0.5° C./0.5 s.

β-Lactamase Activity Assay

Bacterial strains were streaked from frozen LB+glycerol stocks onto LB agar plates and incubate for 24 hours at 37° C. Single colonies were grown overnight at 37° C. in 3 ml of LB. Cultures were diluted 1:1000 in 2 ml LB supplemented with 128 μg/ml meropenem and DNAzyme were added to a final concentration of 1.25 M. Cultures were incubated at 37° C. with continuous shaking for 1 h. Measurement of the β-lactamase activity assay was done using colorimetric kit (abcam), according to the manufacture's protocol.

Viability Assay

HT-29 cells (ATCC) were seeded in 24-well plates at 200,000 cells per well. DNAzyme were added to the medium at different concentrations. After 72 hours, PrestoBlue reagent (A13262, Life Technologies Ltd.) was added as a cell viability indicator.

Galleria mellonella Virulence Assays

Larvae virulence experiments were performed in the facilities of Pharmaseed LTD (Ness Ziona, Israel). G. larvae were obtained from BioSystems Technologies (Exeter, UK) and stored in the shipment boxes at 15° C. for two weeks or at room temperature for immediate use with no requirement of food. Larvae were randomly selected from a large batch for an approximate weight of 250±25 mg. Larvae were separated from sawdust and selected for plumped, lighter colored worms. Darker colored, dehydrated worms, were discarded. Each treatment group (20 larvae per group) contained 10 larvae placed in a 10 cm empty Petri dish (2 dishes per treatment group), on a filter paper.

Bacterial inoculums were prepared from cultures grown overnight at 37° C. in TSB medium (HyLabs, Israel) and washed with sterile 1×PBS. Bacterial suspension concentration was determined by McFarland (Densicheck plus, Biomerieux) and diluted in sterile PBS to receive 10⁹ CFU/mL. Serial dilutions of the bacterial suspensions were made and spots were plated on blood agar plates. The plates were incubated at 37° C. and on the next day CFU was counted in order to determine final injected bacterial concentrations.

Each larva was initially injected with 5×10⁵ cells into the proleg. A second injection to the proleg was done with meropenem (10 μg/g), meropenem+DNAzyme (8.52 μg/g) or sterile PBS. Infected larvae and PBS-injected controls were maintained at 37° C. and checked every 12 hours daily up to one week. Larvae are recorded as dead if they turn grey or black and/or do not respond to touch of head with blunt forceps.

Ex Vivo

Lungs were harvested from 5-10 mice (C57BL/6, 6-8 weeks old) and placed in petri dishes containing DMEM 5% FCS. The tissue was divided into circular pieces 3 mm in diameter with a biopsy punch and transferred to poly-propanol tubes with 3 ml DMEM. DMEM was applied with meropenem and DNAzymes, as indicated. To each respective tube 10 μl of mid-logarithmic Klebsiella pneumoniae culture was added—with three technical repeats for each condition. The plates were incubated at 37° C. for approximately 1 days (24 hours), washed twice with PBS.

For CFU of bacteria infecting ex vivo tissue culture: individual punches or their growth media were collected, Punches were resuspended in 1 ml PBS (Biological Industries), and free-living bacteria were pelleted and resuspended in PBS at the same volume. Samples were thoroughly vortexed (5 min). The samples were then mildly manually crushed using 1.5 ml pestle. In all cases, to determine the number of viable cells, samples were serially diluted in PBS, plated on LB plates, and colonies were counted after incubation at 37° C. overnight.

KP Infected Thigh Muscle Model in Neutropenic Mice

Neutropenia induction: The experiments were performed on female ICR mice age 9-10 weeks. At day (−4) neutropenia induction was performed by cyclophosphamide intraperitoneal (IP) injection at 150 mg/kg and a volume of 10 mL/kg. At day (−1) neutropenia maintenance was performed by cyclophosphamide IP injection at 100 mg/kg and a volume of 10 mL/kg.

Bacterial suspension preparation: At day (−4) KP (ATCC® BAA-1705™) bacteria was streaked out from −80° C. glycerol stock onto LB medium and incubated at 37° C. for 20 hours and then stored at 2-8° C. At day (−1) single colonies of the KP (ATCC® BAA-1705™ bacteria) were picked by using an inoculation loop (Greiner, 731175) and suspended in 20 mL Tryptic Soy broth (TSB) (Hylabs, BIO-MO11) in Erlenmeyer and incubated at 37° C. overnight (=starter) with shaking at 180 rpm. In the morning of dosing day, bacterial starter was diluted in fresh TSB (total of 20 mL) and incubated for about 1-3 hours at 37° C. Bacterial culture was centrifuged at 3500 g for 10 minutes at RT and the pellet was resuspended in sterile PBS. Bacterial suspension concentration was determined by McFarland (Densicheck plus, Biomerieux) and diluted in sterile PBS to a concentration of 2.2×10⁷ CFU/mL.

Inoculum and treatment: On day 1 each mouse was injected intramuscular (IM) with 50 μL of bacterial suspension in each thigh (both legs per animal), Meropenem was injected subcutaneous (SC) at a dose volume of 15 mL/kg (240 mg/kg) while the DNAzyme KPC-337 was injected SC at a dose volume of 2.5 mL/kg (16, 24 or 32 mg/kg) approximately every 6 hours during the workday and every 8 hours at night, till the end of the study.

Termination and Necropsy: Upon termination, animals were sacrificed by CO₂ asphyxiation. Muscle tissues from both femur and pelvic bones (thigh) both legs were harvested. Tissues were weighed and homogenized in 3 mL of ice-cold PBS using gentleMACS™ Dissociator.

Bacterial burden determination: Homogenate samples dilutions were plated on LB agar plates at various dilutions to determine the colony forming units (CFUs). Plates were incubated overnight (at 37° C.), and bacterial burden was calculated and expressed as CFUs/gram of tissue.

### Example 1

### Design of DNAzymes Targeting Bacterial Resistance Genes

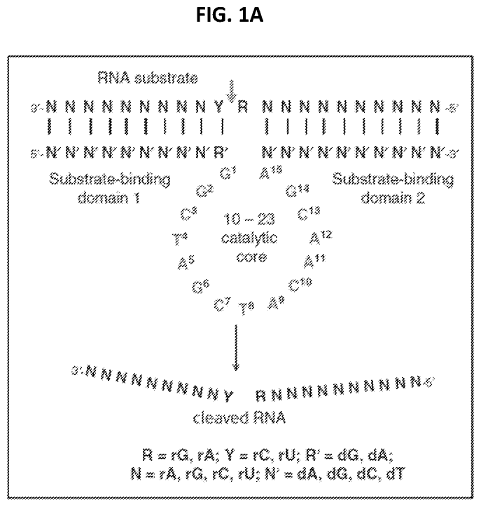

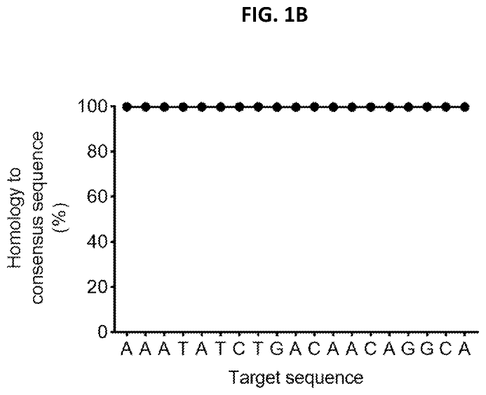

The potential of catalytic deoxyribozymes (i.e. DNAzymes) that are capable of hydrolyzing specifically and efficiently RNA transcripts of resistance genes and restore the antibiotic susceptibility of resistant isolates of bacteria was examined. This was carried out by designing DNAzymes that specifically cleave selective RNA transcripts that based on the literature confer antibiotic resistance (Tables 1 and 2). The DNAzymes consist of a catalytic core flanked by two arms that recognize its RNA target through Watson Crick base pairing and cleave RNA in a specific phosphodiester linkage (FIG. 1A).

### Example 2

### DNAzyme Targeting KPC Gene in Klebsiella pneumoniae

One DNAzyme (KPC-337, as set forth in SEQ ID NO: 1) was designed that targets a conserved region in RNA transcripts of the gene bla carbapenemase of clinically relevant strains of Klebsiella pneumoniae (ec number 3.5.2.6, uniport ID Q9F663). In total, 30% (3845/12539) sequenced isolate of Klebsiella pneumoniae have the annotation of bla-KPC in which the target region (AAATATCTGACAACAGGC, bases 345-363, SEQ ID NO: 27) is conserved in 99.6% of the isolates (FIG. 1B).

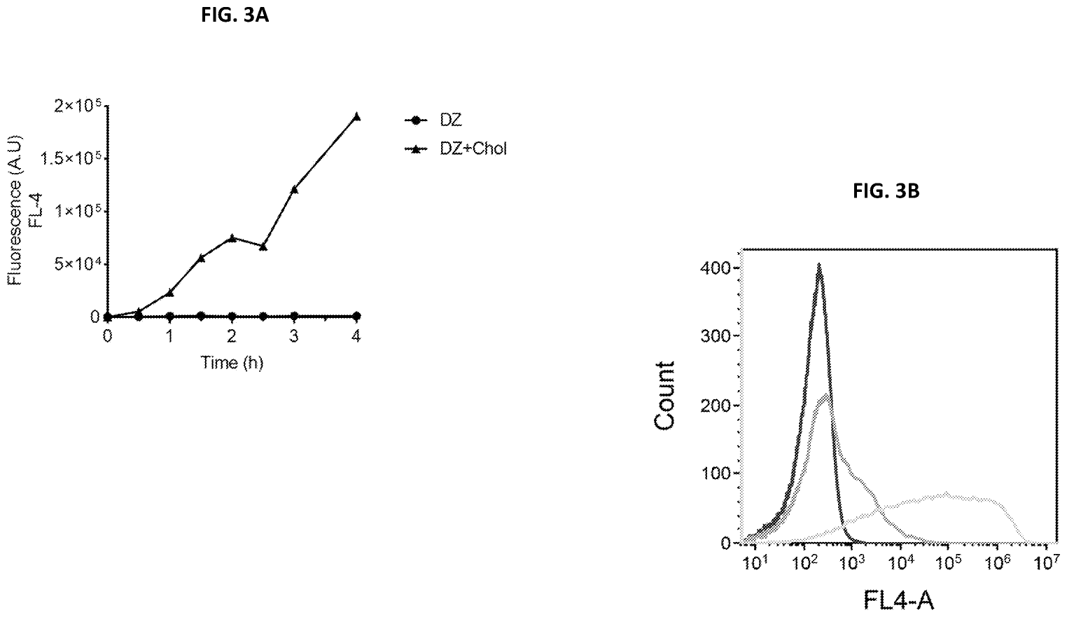

The first obstacle in applying DNAzyme as antibacterial agents is the delivery of DNAzymes into bacterial cells efficiently. DNAzyme have to overcome multiple physiological barriers from the administration site to the intracellular of the bacteria. The major hurdle for internalization of DNAzymes to the bacterial cells is the cell wall. Notably, the outer membrane of a gram-negative bacteria, which is absent in gram-positive bacteria, is the main reason for resistance to a wide range of antibiotics due to its hydrophobic nature which physically blocks the diffusion of some antibiotics. Conjugation of cholesterol to DNAzymes facilitated the uptake of DNAzymes into Klebsiella pneumoniae strain ATCC® BAA-1705™ in media mimicking the in vivo conditions in the presence of sub toxic concentration of meropenem (FIGS. 3A-B).

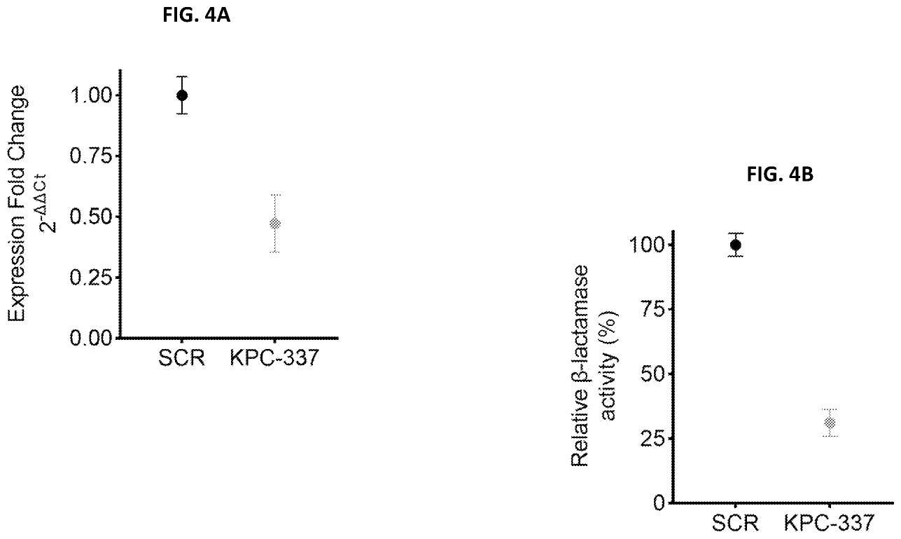

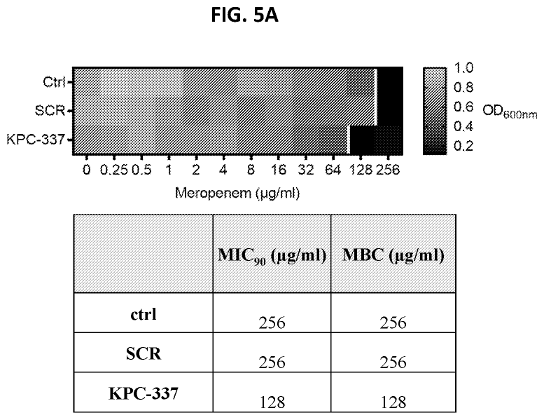

To test the bioactivity of the DNAzyme KPC-337, the RNA and protein levels of bla-KPC were measured following combined treatment with sub-toxic concentration of meropenem and the DNAzyme KPC-337. Addition of the DNAzyme KPC-337 to bacterial culture of Klebsiella pneumoniae strain ATCC® BAA-1705™ reduced the transcript levels of bla-KPC by two fold (FIG. 4A) and the β-lactamase activity by 70% (FIG. 4B). All experiments included a control that consisted of the same nucleotides and the cholesterol-TEG at the 3′ as the DNAzyme KPC-337 but in a random non catalytic order designated as “scramble” (SCR). The SCR control did not changed the RNA levels nor the β-lactamase activity compared to untreated control. This indicates that the DNAzyme is capable of entering the bacteria, binding to and specifically cleaving the intracellular RNA transcripts.

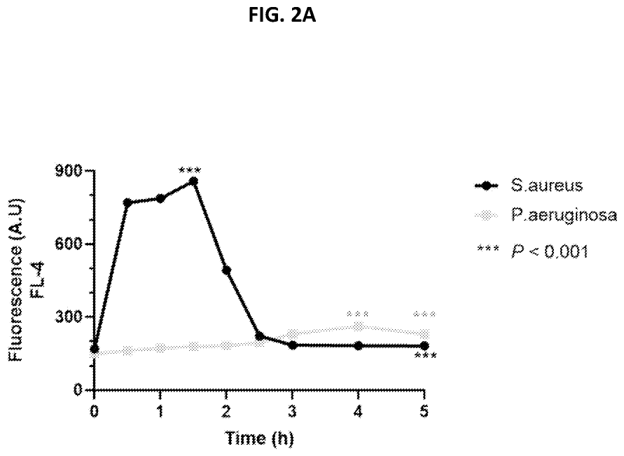

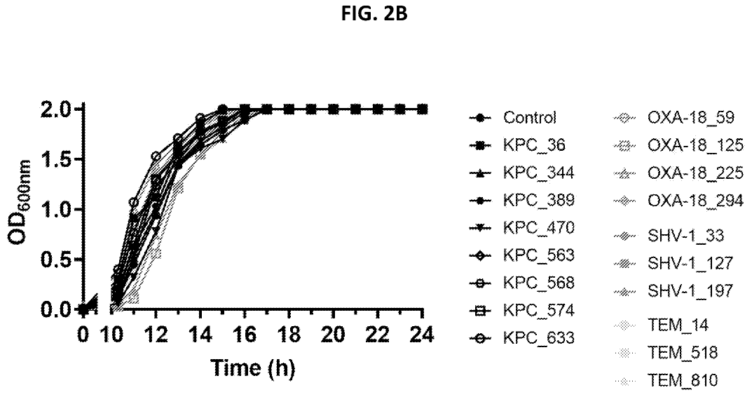

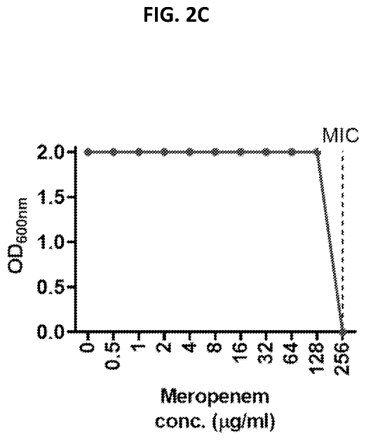

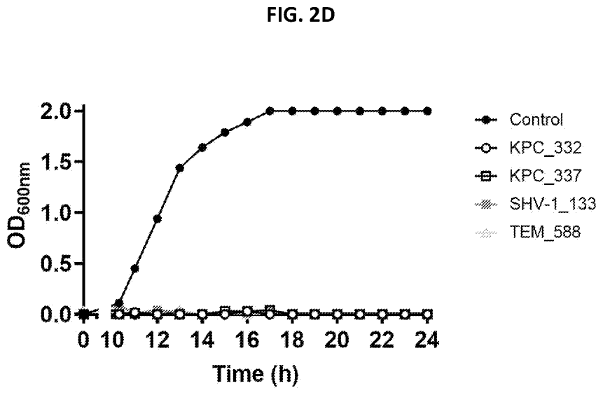

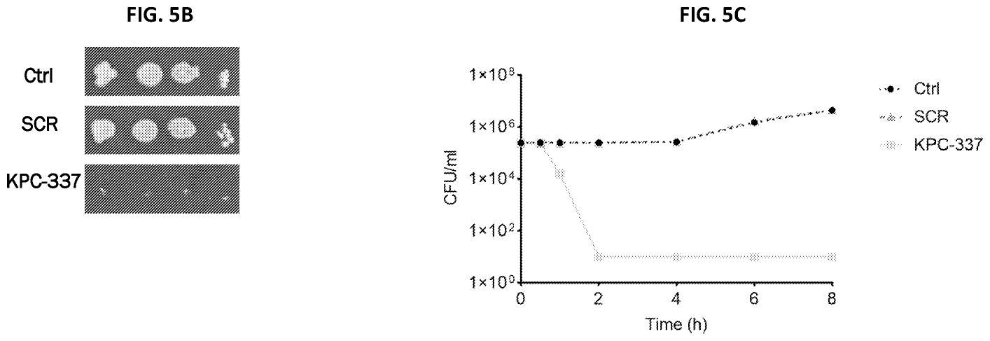

The DNAzyme (KPC-337) targets a conserved region in RNA transcripts of the gene bla carbapenemase that confers resistance to carbapenem antibiotics in carbapenem-resistant Klebsiella pneumoniae (CRKP). Thus, addition of the DNAzyme KPC-337 to bacterial culture of Klebsiella pneumoniae ATCC® BAA-1705™ grown with increasing antibiotic concentrations reduced the MIC levels by two fold (from 256 μg/ml to 128 μg/ml, FIGS. 2C and 5A). The combined treatment of meropenem and KPC-337 is bactericidal, thus reducing the viability of the bacterial cells (FIGS. 5B-C).

Additional DNAzymes that target resistance genes (e.g. KPC, TEM, SHV-1 and OXA-18, e.g. as set forth in SEQ ID Nos: 2-4, 35-53) were tested for sensitization of a bacterial culture of Klebsiella pneumoniae ATCC® BAA-1705™ and four of the tested DNAzymes had an inhibitory effect on the bacterial growth (FIGS. 2B and 2D).

### Example 3

### Ex Vivo Effect of a DNAzyme Targeting KPC Gene in Klebsiella pneumoniae

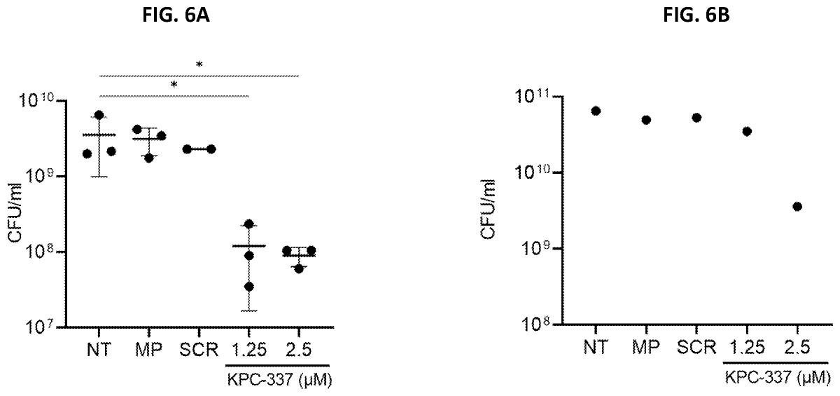

Klebsiella pneumoniae is a vital pathogen of community and hospital-acquired pneumonia. Thus a lung ex-vivo infection model was used to test the effect of the DNAzymes during the initial infection stages. Klebsiella pneumoniae is able to colonize the lung tissue whereas treatment with sub-toxic levels of meropenem with KPC-337 reduced the capacity of the bacteria to infect and colonize lung tissue by approximately 1.5-2 fold (FIG. 6A). In addition, a further reduction of the free-living cells that remained in the growth media was also observed (approximately 1-1.5 fold change, FIG. 6B). Importantly, treatment with meropenem alone or in combination with the scramble control did not reduced the bacterial load in the tissue or in the growth media (FIG. 6B).

### Example 4

In vivo effect of a DNAzyme targeting KPC gene in Klebsiella pneumoniae The implementation of infection in vivo models that approximate human disease is essential for testing new therapies before entering into clinical stages.

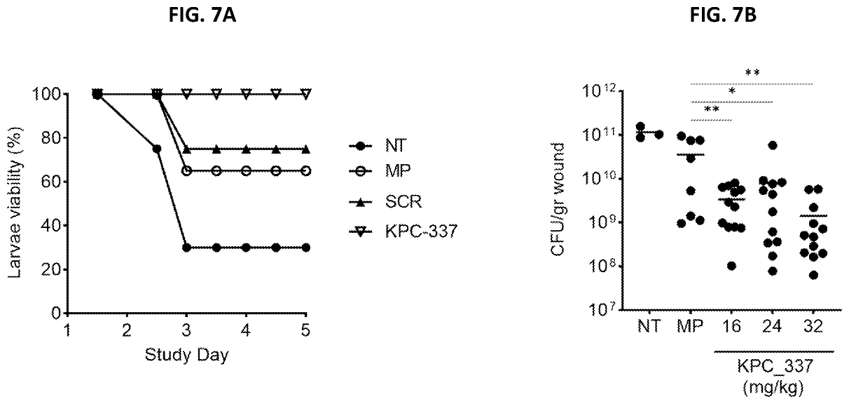

Wax moth Galleria mellonella has been utilized to study key virulence mechanisms in Klebsiella sp., such as cell death associated with bacterial replication, avoidance of phagocytosis by phagocytes, the attenuation of host defense responses, and the production of antimicrobial factors. G. mellonella model is useful for assessing pathogenic potential of Klebsiella pneumoniae and for testing new therapies. Bacterial infection of larvae of G. mellonella with Klebsiella pneumoniae strain ATCC® BAA-1705™ reduced the viability of the larvae by 75%, while treatment with sub-toxic concentration of meropenem (10 μg/g) alone or with combination with the scramble control reduced the viability by 40% and 35%, respectively. Treatment with sub-toxic concentration of meropenem with KPC-337 reduced the toxicity of the infection and the larvae viability remained 100% (FIG. 7A). Thus, the combined treatment is effective for elimination of infection caused by resistant Klebsiella pneumoniae in the larvae model.

Next, the effectiveness of the combined therapy was examined in the neutropenic thigh infection model. The thigh infection model provides a sensitive experimental system for initial studies of antimicrobial efficacy in a mammalian system. This model is the most standardized for the evaluation of antimicrobial interaction combined with antimicrobial pharmacokinetics assessed in serum and tissues samples. Accordingly, using the thigh model the animals were infected with Klebsiella pneumoniae in both thighs and were treated three times a day (TID) with 240 mg/kg meropenem alone or combined with different amounts of KPC-337 (16, 24 or 32 mg/kg) via subcutaneous administration. The results illustrated that treatment with meropenem alone slightly reduced the bacterial load in the infection site (not significant, FIG. 7B). However combined treatment of meropenem with KPC-337 reduced the bacterial load significantly (by 2-3 log fold) in the infection site (FIG. 7B). Thus, the combined therapy of antibiotic with KPC-337 rendered susceptibility in a murine in vivo model.

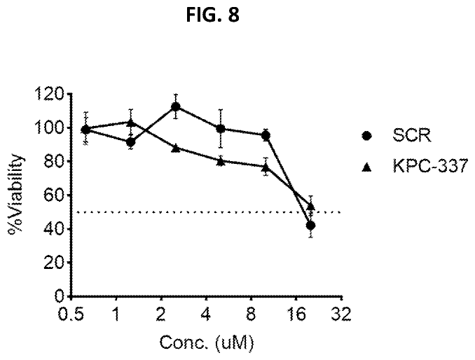

The toxicity of the DNAzyme on human cell line was further tested in a HT-29 cell line (human colorectal adenocarcinoma). The IC₅₀ of toxicity of the KPC-337 and the scramble control were both 20 μM (FIG. 8). This implies that the toxicity was not due to the sequence of the DNAzyme. Importantly, the DNAzyme KPC-337 was not toxic to human cells in bioactive concentration (e.g. <5 μM).

### Example 5

### Therapeutic In Vivo Effect of a DNAzyme Targeting Klebsiella pneumoniae Genes

Klebsiella pneumoniae is responsible for a growing proportion of nosocomial infections, including pneumonia, urinary tract infection and sepsis. Therefore, the effectivity of a combination therapy comprising a DNAzyme (e.g. KPC-337) and an antibiotic (e.g. meropenem) is expanded to additional in vivo infection murine models including pulmonary infection model, urinary tract infection model and sepsis.

### Example 6

### DNAzymes Targeting Methicillin-Resistant Staphylococcus aureus (MRSA)

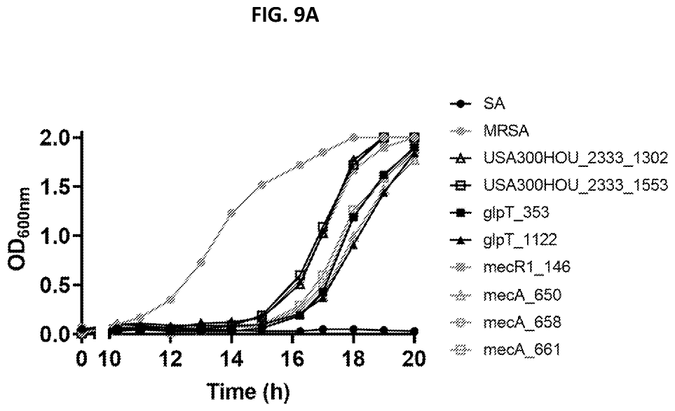

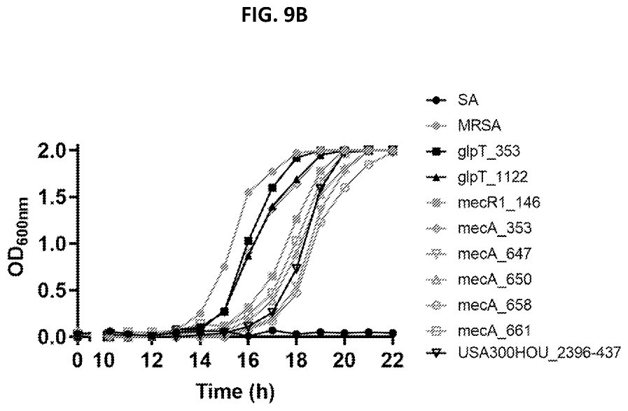

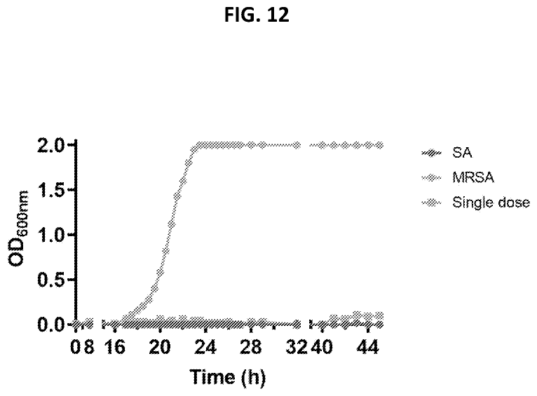

DNAzymes (as set forth in SEQ ID NO:5-26) were designed to target a conserved region in RNA transcripts of the resistance genes mecA, mecR1, glpT, femA and USA300HOU. As described above, the first obstacle in applying DNAzyme as antibacterial agents is the delivery of DNAzymes into bacterial cells efficiently. Conjugation of cholesterol to DNAzymes facilitate the uptake of DNAzymes into the gram positive methicillin-resistant Staphylococcus aureus strain (MRSA) ATCC® BAA-1705™ and in a minor degree to a gram negative Pseudomonas aeruginosa ATCC® BAA-3105™ (FIG. 2A). The addition of the DNAzyme to a bacterial culture of methicillin-resistant Staphylococcus aureus strain ATCC® BAA-1705™ inoculated with a sub-toxic concentration of cefoxitin delayed the growth of the bacteria, thus increasing the susceptibility of the bacteria to the antibiotic (FIGS. 9A-B). A remarkable inhibitory effect was observed using a single dose treatment of the femA targeting DNAzyme, femA-545 (as set forth in SEQ ID NO: 25) together with cefoxitin, which inhibited the growth of the bacterial culture for 44 hours (FIG. 12).

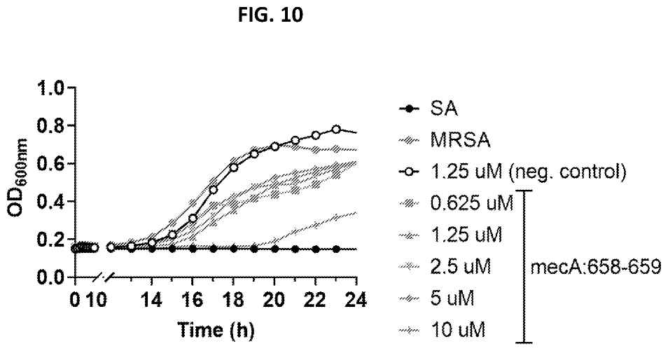

The inhibitory effect of the DNAzyme was illustrated to be dose dependent wherein an increase in the therapeutic dose of the DNAzymes, increased the delay of the bacterial growth (FIG. 10).

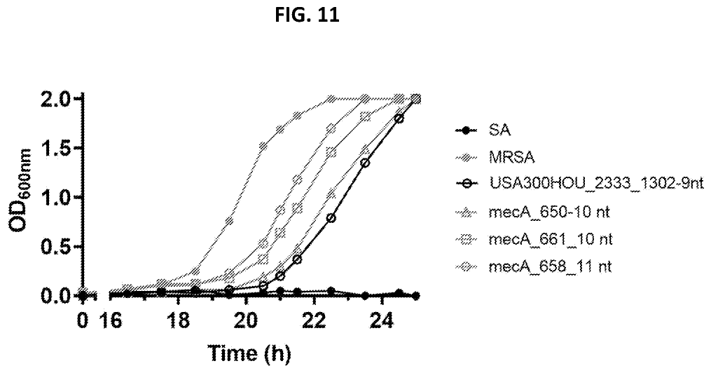

As mentioned, DNAzymes consist of a catalytic core flanked by two arms that recognize its RNA target through Watson Crick base pairing. The strength of the pairing of the DNAzymes to the RNA target is depending upon the Tm of the binding. Thus, extension of the length of the flanking arms was expected to increase the Tm and the pairing of the DNAzymes to the target RNA. Indeed, optimizing the flanking arms lengths of selected DNAzymes resulted in increase in the lag of the bacteria growth (FIGS. 10 and 11).

### Example 7

### Improved Delivery of DNAzymes into Bacterial Cells

In order to enhance the delivery of the DNAzymes into bacterial strains, DNAzymes are designed to comprise modifications on the oligonucleotide and/or by utilizing lipid nanoparticles that can selectively enter only bacterial cells. This method improves the specificity of delivery to the target bacteria and can further decrease the toxicity of treatment. In addition, improved targeting into bacterial cells increases the intracellular DNAzyme copy numbers that in turn improve cleavage efficiency of the target in the bacteria.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An oligonucleotide comprising a nucleic acid sequence of at least one DNAzyme, wherein at least one target of said at least one DNAzyme is a bacterial target selected from the group consisting of:
Carbapenemase: (5R)-carbapenem-3-carboxylate synthase: EC: 1.14.20.3,
USA300HOU;
mecA: penicillin-binding protein 2A;
mecR1: beta-lactamase-sensing transmembrane signaling protein;
glpT: glycerol-3-phosphate transporter;
femA: aminoacyltransferase: EC: 2.3.2.17; and
ESBL: Extended-spectrum beta-lactamase.

2. The oligonucleotide of claim 1, wherein
when said target is Carbapenemase, said Carbpenemase is KPC: Carbapenem-hydrolyzing beta-lactamase, EC: 3.5.2.6; or
when said target is ESBL, said ESBL is selected from the group consisting of:
SHV: sulfhydryl variable: beta-lactamase: EC: 3.5.2.6,
TEM: extended spectrum beta-lactamase: EC: 3.5.2.6,
OXA: oxacillin hydrolyzing enzyme: EC: 3.5.2.6, and
CTX-M: beta-lactamase: EC: 3.5.2.6.

3. (canceled)

4. (canceled)

5. The oligonucleotide of claim 1, wherein the at least one DNAzyme comprises a plurality of DNAzymes, and wherein said oligonucleotide comprises a cleavable nucleic acid sequence between each pair of said plurality of DNAzymes, and wherein optionally, said oligonucleotide comprises a promoter sequence between each pair of said plurality of DNAzymes.

6. (canceled)

7. (canceled)

8. (canceled)

9. (canceled)

10. The oligonucleotide of claim 1, wherein said nucleic acid sequence comprises at least one modification, said modification comprising a modification in the catalytic core of said at least one DNAzyme or a modification in a binding arm of the at least one DNAzyme, wherein said modification comprises
an insertion, a deletion, a substitution or a point mutation of at least one nucleic acid;
an addition of one or more nucleotides on a 5′ and/or 3′ terminus of said nucleic acid sequence; or
a base modification, a sugar modification, or an internucleotide linkage modification, or a combination thereof;
or any combination thereof.

11. (canceled)

12. (canceled)

13. (canceled)

14. (canceled)

15. (canceled)

16. (canceled)

17. The oligonucleotide of claim 10, wherein when said modification comprises a base modification, a sugar modification, or an internucleotide linkage modification, or a combination thereof, said modification is selected from the group consisting of locked nucleic acids (LNA), phosphorothioate, 2-O-fluor, 2-O-methyl, 2-O-methoxyethyl, methylcytosine, 2-fluoro, and a 2-Fluoroarabinooligonucleotides.

18. The oligonucleotide of claim 1, wherein said nucleic acid sequence is set forth in any one of SEQ ID NOs: 1-26 or 35-53, or wherein said nucleic acid sequence is at least 80% identical to the oligonucleotide sequence set forth in any one of SEQ ID NOs: 1-26 or 35-53.

19. (canceled)

20. (canceled)

21. (canceled)

22. (canceled)

23. (canceled)

24. (canceled)

25. The oligonucleotide of claim 1, said oligonucleotide further comprising a permeability enhancing moiety attached to a nucleotide of the at least one DNAzyme, wherein the permeability enhancing moiety is a cholesterol moiety, a cell penetrating peptide, a lipid nanoparticle, or a viral capsid.

26. (canceled)

27. (canceled)

28. (canceled)

29. The oligonucleotide of claim 1, wherein the bacteria is selected from the group consisting of a Enterococcus faecium, a Staphylococcus aureus, a Klebsiella pneumoniae, an Acinetobacter baumannii, a Pseudomonas aeruginosa, and an Enterobacter.

30. (canceled)

31. A pharmaceutical composition comprising the oligonucleotide of claim 1, and a pharmaceutically acceptable carrier or diluent.

32. The pharmaceutical composition of claim 31, and further comprising an antibiotic.

33. The composition of claim 32, wherein said oligonucleotide and said antibiotic are in a co-formulation or are in separate formulations.

34. The composition of claim 32, wherein said antibiotic is a β-lactam or said antibiotic is selected from the group consisting of penicillin, methicillin, oxacillin, cephalosporin, aztreonam, cefoxitin, carbapenem, imipenem, and meropenem.

35. A method of treating or preventing a bacterial infection in a human subject in need thereof, the method comprising administering to the subject the oligonucleotide of claim 1 and an antibiotic, thereby treating or preventing the bacterial infection in the subject.

36. (canceled)

37. The method of claim 35, wherein said oligonucleotide and said antibiotic are in separate formulations or are in a co-formulation.

38. (canceled)

39. (canceled)

40. The method of claim 35, wherein said antibiotic is a β-lactam or said antibiotic is selected from the group consisting of penicillin, methicillin, oxacillin, cephalosporin, aztreonam, cefoxitin, carbapenem, imipenem, and meropenem.

41. A surface coated with the oligonucleotide of claim 1, said surface optionally further coated with an antibiotic.

42. The surface of claim 41, wherein said antibiotic is a β-lactam or said antibiotic is selected from the group consisting of penicillin, methicillin, oxacillin, cephalosporin, aztreonam, cefoxitin, carbapenem, imipenem, and meropenem.

43. (canceled)

44. The oligonucleotide of claim 1, comprising ribonucleotides, deoxyribonucleotides, or combination thereof.

45. The oligonucleotide of claim 25, wherein permeability enhancing moiety is attached at the 3′ end of the nucleic acid coding for said DNAzyme, and said permeability enhancing moiety is cholesterol—TEG (triethylene glycol).

46. The method of claim 35, wherein said oligonucleotide further comprises a permeability enhancing moiety attached to a nucleotide of the at least one DNAzyme, wherein the permeability enhancing moiety is a cholesterol moiety, a cell penetrating peptide, a lipid nanoparticle, or a viral capsid.

47. The method of claim 46, wherein the permeability enhancing moiety is attached at the 3′ end of the nucleic acid coding for said DNAzyme, and said permeability enhancing moiety is cholesterol—TEG (triethylene glycol).

48. The pharmaceutical composition of claim 31, wherein said oligonucleotide further comprises a permeability enhancing moiety attached to a nucleotide of the at least one DNAzyme, wherein the permeability enhancing moiety is a cholesterol moiety, a cell penetrating peptide, a lipid nanoparticle, or a viral capsid.

49. The pharmaceutical composition of claim 48, wherein the permeability enhancing moiety is attached at the 3′ end of the nucleic acid coding for said DNAzyme, and said permeability enhancing moiety is cholesterol—TEG (triethylene glycol).
